(19) United States
(12) Patent Application Publication
(10) Pub. No.: US 2026/0026745 A1
(43) Pub. Date: Jan. 29, 2026

(54) TREATMENT SYSTEMS FOR NOCTURNAL HYPOXIA IN CARDIOVASCULAR PATIENTS

(71) Applicant: RMX, LLC, San Francisco, CA (US)

(72) Inventor: Amir J. TEHRANI, San Francisco, CA (US)

(73) Assignee: RMX, LLC, San Francisco, CA (US)

(21) Appl. No.: 19/276,520

(22) Filed: Jul. 22, 2025

Related U.S. Application Data
(60) Provisional application No. 63/675,904, filed on Jul. 26, 2024.

(51) Int. Cl. A61B 5/00; A61B 5/0205; A61B 5/024; A61B 5/053; A61B 5/08; A61B 5/11; A61B 5/1455; A61B 5/318; A61B 5/369; A61N 1/36

(52) U.S. Cl. CPC A61B 5/4836; A61B 5/0205; A61B 5/02405; A61B 5/053; A61B 5/0816; A61B 5/11; A61B 5/14551; A61B 5/318; A61B 5/369; A61B 5/4818; A61B 5/686; A61B 5/7246; A61N 1/36057; A61N 1/3611; A61N 1/36139; A61B 2560/0468; A61B 2562/0219; A61B 2562/0247

(57) ABSTRACT

Treatment systems for nocturnal hypoxia in cardiovascular patients are described herein. Generally, the system may include one or more sensors configured to be implanted within a subject and a controller in communication with the one or more oximetry sensors, wherein the controller is programmed to predict a change in oxygen saturation indicative of hypoxia in the subject by identifying one or more patient-specific features from a polysomnography of the subject and correlating the one or more patient-specific features to data received from the one or more sensors.

### CROSS-REFERENCE TO RELATED APPLICATIONS

This application claims the benefit of priority to U.S. Prov. App. 63/675,904 filed Jul. 26, 2024, which is incorporated herein by reference in its entirety.

### FIELD OF THE INVENTION

The present invention relates to devices and methods for treating a variety of conditions, disorders or diseases using a sheath system with deployable mapping electrodes. The present invention also relates to maintaining a personalized and stable level of oxygen saturation (SaO2) for a particular patient through minute ventilation modulation as well as maintaining respiratory functions within the body within the stable range. This could be during wakefulness or sleep. There are multiple causes of reduction or unstable SaO2 including respiratory and cardiovascular disorders such as COPD, Sleep Apnea, Coronary Artery Disease (CAD), atrial fibrillation (AFIB), Chronic Heart Failure (CHF), Obesity hypoventilation syndrome, and several others.

The present invention includes device and methods in detecting and predicting changes in SaO2 and utilization of minute ventilation modulation or other respiratory functions to minimize occurrence of hypoxia in patients. Nocturnal hypoxia is an independent risk factor to increase mortality and morbidity in patients such as cardiovascular patients.

### BACKGROUND OF THE INVENTION

Sleep apnea and hypopnea are breathing disorders that occur during periods of sleep where an intermittent cessation or reduction of ventilation during sleep results in a decrease in blood oxygen levels (hypoxia) and an increase in CO2 (hypercapnia). The long term effects of these physiological conditions are associated with the development of various disorders including cardiac arrhythmias, congestive heart failure, cardiac ischemia, hypertension, and heart disease, amongst other conditions.

CPAP is the most common treatment for obstructive sleep apnea and has been proposed for central sleep apnea. CPAP requires an external device and patient compliance. In addition, its cardiovascular effects are currently unclear and some researchers believe that it can exacerbate heart failure in some patients, particularly where positive forced pressure has a negative effect on a heart failure patient, such as, for example, in patients where a reduced ventricular filling would significantly reduce cardiac output. Diaphragm stimulation has been proposed to treat central sleep apnea by stimulating when apnea has occurred. However, the stimulation is provided after the apnea event has occurred rather than preventing the apnea event. Hypoglossal nerve stimulation has been proposed to treat obstructive sleep apnea by increasing patency in the upper airway to allow respiration.

Hypoxia is a state in which oxygen in not available in sufficient amount at the tissue level to maintain adequate homeostasis; this can result from inadequate oxygen delivery to the tissues either due to low blood supply or low oxygen content in the blood (hypoxemia). Research points to nocturnal hypoxia and hypoxemia as independent risk factor for cardiovascular mortality and morbidity among patients with sleep disorder breathing, cardiovascular disease such as AFIB, heart failure, obesity, and COPD (chronic obstructive pulmonary disorder).

In addition to positive pressure therapies, weight loss, bariatric surgery, oxygen therapy, and pharmaceuticals have not shown a significant impact on the CV endpoints and outcomes.

Phrenic nerve stimulation to treat CSA has shown to treat majority of CSA episodes and improve oxygen saturation. However, the device did not treat the OSA episodes in the same patients and was not powered to measure CV endpoints.

Thus, there is no current viable therapy to directly treat both forms of hypoxia and particular outcomes such as cardiovascular outcomes. What are needed are treatments for the root cause of cardiovascular risks in patients experiencing nocturnal hypoxia whether from desaturation caused by apnea or non-specific causes.

### SUMMARY OF THE INVENTION

A neurostimulation lead system may be comprised of a commercial lead mounted and connected to a flexible frame fabricated from nitinol or other biocompatible material where the frame may be inserted and advanced via flexible catheter body through a vessel into proximity of a nerve to be stimulated. The frame can be constructed of nitinol wires where the nitinol wires act as a “guidewire” which can be inserted through a lumen. Alternatively, the lead can be placed and fastened to the frame such that as the frame expands, the lead may adhere to the vessel wall in the intended position where the lead is facing the targeted nerve through the vessel. The frame and lead system may be housed or inserted inside a sheath or mapping sheath. Specific markers and labels on the sheath and sheath catheter may provide orientation of the lead and electrodes in reference to the targeted nerve. As the sheath is pulled away (e.g., pulled proximally), the nitinol frame may slowly expand in the intended location within the vessel and orientation relative to the nerve to be stimulated. The frame/lead system can also be collapsed by pushing the sheath distally over the frame and repositioned and released again and/or removed from the vessel.

One variation of an endovascular lead apparatus may generally comprise an elongate body having a proximal portion and a distal portion, one or more electrodes positioned along a first side of the distal portion, a tray positioned along a second side of the distal portion opposite to the first side, and one or more frame members positioned along the tray and which are reconfigurable from a low-profile delivery configuration to an expanded deployed configuration, wherein expansion of the one or more frame members to the expanded deployed configuration reconfigures the distal portion into contact against a tissue wall for energy delivery via the one or more electrodes.

One variation of a method of stimulating a nerve may generally comprise advancing an elongate body within a vessel and into proximity of a nerve body to be stimulated, confirming a location of the nerve body via one or more mapping electrodes positioned along the elongate body, exposing a distal end of the elongate body such that one or more frame members positioned along the distal end reconfigure to urge a first side of the distal portion into contact against an inner surface of the vessel, and actuating one or more treatment electrodes positioned along the first side to deliver a treatment stimulation through the inner surface and into the nerve body.

In accordance with the invention, stimulation is provided to the diaphragm or phrenic nerve to elicit a diaphragm response to thereby provide a therapeutic effect for a heart failure or other cardiac or cardiovascular patient.

In another variation, the electrode can be placed surgically near or around the phrenic nerve as described in U.S. Pat. No. 8,467,876. A cuff electrode with 2-4 contacts can be placed around one or both phrenic nerves. The lead system will be tunneled through the neck to an IPG with sensing and stimulation electronics, software, and algorithms.

In yet another variation, an injectable lead with 2-4 or more electrode contacts can be placed near one or both phrenic nerves under imaging guidance such as ultrasound. The injectable lead system can be activated with a wearable sensing and stimulation system. The wearable will transmit stimulation signals upon sensing the hypoxia prediction or detection.

In accordance with one aspect of the invention, stimulation to elicit a diaphragm response is provided to increase or normalize lung volume and in particular to increase functional residual capacity. It is believed that stimulation to increase or to normalize lung volume or functional residual capacity may have one or more effects that may be therapeutic to cardiovascular or heart failure patients. Normalizing herein may include for example, bringing a physiological parameter into a normal or healthy region for patients or for a particular patient, or to a level appropriate for a condition or state of a patient.

In accordance with another aspect of the invention stimulation is provided to control breathing to reduce respiration rate and thereby reduce hypertension, reduce sympathetic nerve bias, and/or provide improved blood gas levels.

In accordance with another aspect of the invention stimulation is provided to control minute ventilation to therapeutically effect blood gas levels.

In accordance with another aspect of the invention, stimulation is provided to create a deep inspiration or an increased tidal volume to thereby reduce sympathetic nerve bias, improve blood gas levels, stimulate reflexes for example the Hering-Bruer reflex related to activating stretch receptors, increase lung volume, normalize or reset breathing or provide other beneficial therapies to improve cardiovascular function or heart failure condition.

In accordance with another aspect of the invention stimulation may be provided to modulate intrathoracic pressure to thereby produce a therapeutic effect. Modulation of intrathoracic pressure is expected to impact sympathetic activation and improve heart conditions. It is known that in chronic heart failure settings, increased cardiac filling pressures and/or pulmonary pressures may cause a direct or indirect reflex increase in sympathetic efferent outflow to the heart. Therefore, a sustained reduction or average reduction in intrathoracic pressure through modulation of intrathoracic pressure could have an opposite effect and reduce sympathetic efferent outflow to the heart. One would expect to reduce norepinephrine spillover through intrathoracic pressure modulation which is beneficial to the heart failure patient. Parker et al. used a lower body pressure chamber to show certain reduction in body pressure leads to reduction in cardiac filling pressures leading to reduction in norepinephrine spillover in acute setting. Longer term application this therapy has potential to improve the heart failure and also remodel the cardiac tissue.

The devices and methods described may achieve similar results through various modes of phrenic nerve and/or diaphragm stimulation. These stimulation modalities include low-level stimulation overlapped with patient intrinsic breathing, diaphragm bias, breath augmentation, increase in tidal volume, increase in inspiration duration, deep inspiration, breathing entrainment, manipulation of exhalation period and volume, increasing and maintain resting lung volume or functional residual capacity, sustained stimulation during inspiration and/or exhalation, continuous stimulation, stimulation synchronized with respiratory cycles, or cardiac cycles, and/or duty-cycled type stimulation based on a percentage of time, for example, 20% of the time during the day or night and when patient is sleep or awake. The duty-cycled type stimulation could be synchronized to a respiratory cycle or not.

In accordance with another aspect of the invention, stimulation may be provided to modulate intrathoracic pressure targeting a sustained reduction in average central venous pressure to effectively reduce right arterial and right ventricular pressures and pulmonary wedge pressure. Reduction in right ventricular pressure in heart failure patients leads to increase stroke volume and therefore cardiac output. The sustained increase in cardiac output though reduction in filling pressure will lead to reductions in pulmonary congestion which is a major reason for acute heart failure decompensation and therefore hospitalization. Some other hemodynamic effects of this stimulation could be reduction in heart rate as results of increased in cardiac output and/or reduction in filling pressures.

In accordance with another aspect of the invention, stimulation may be provided to modulate intrathoracic pressure targeting a sustained or incremental reduction in renal pressure or the average renal pressure to improve kidney function and filtration. Abnormal renal function is common in acute and chronic heart failure. It is expected a change in blood volume, cardiac filling pressures, central venous pressure, atrial or ventricular pressures, cardiac output, and/or hemodynamics intervention could lead to improvement of renal function. Intrathoracic pressure modulation could have an impact on pressure within inferior and superior vena cava as well as central venous pressures. Activation of renal sympathetic activity through modulation and manipulation of these pressures could have an impact on kidney pressure and blood transfer rate and ultimately kidney glomerular filtration rate (GFR) and leading to reduction in kidney failure as well as reducing congestion or blood backing up into the lungs through increased filtration. Any of the mentioned phrenic nerve or diaphragm stimulation modalities included in this application could be applied at various situations depending on the need of the patient and sensed parameters. Literature has shown that elevated cardiac filling pressures are associated with reduced GFR. Therefore the present invention tries to reduce cardiac filling pressure through phrenic nerve stimulation and to increase GFR.

In accordance with another aspect of the invention, intrathoracic pressure modulation could be used to treat patients with pulmonary hypertension. Pulmonary hypertension is result of increased pulmonary pressures. Reduction or modulation of intrathoracic pressure could lead to reduction or treatment of pulmonary hypertension.

In accordance with another aspect of the invention the stimulation could be activated by the patient using an external device. The stimulation could be also activated by sensing increased physical activity through an activity sensor or increased in heart rate or respiration rate or other mechanism indicating need for supplemental cardiac output or reduction in filling pressures. For example, a thoracic or lungs impedance sensor or a list of sensors including in the referenced publication cold be used to activate stimulation to deliver therapy to improve hemodynamics.

In accordance with another aspect of the invention stimulation is provided to reduce breathing disorders to thereby improve condition of a heart failure patient.

In accordance with another aspect of the invention a combined cardiac rhythm management device including leadless devices and diaphragm/phrenic nerve stimulation device is provided to provide an enhanced combined treatment device.

In accordance with another aspect of the invention, leadless phrenic nerve electrodes could be injected, delivered, or placed in the vicinity of the phrenic nerve and stimulation cold be performed through an external or integral pulse generator. The sensor or sensors to synchronize the stimulation could be also internal or external to the body.

In accordance with another aspect of the invention a combined vagal nerve, hypoglossal nerve, or cardiac plexus stimulation management device and diaphragm/phrenic nerve stimulation device is provided to provide an enhanced combined treatment device.

The system may also be utilized to provide a continuous or synchronized low level stimulation to the phrenic nerve or diaphragm overlapped with the patient's own intrinsic breathing to reduce an intrathoracic pressure and improve cardiac output. The patient's SaO2 levels may also be improved and the heart and respiration rates may be reduced.

Various mapping and/or neurostimulating electrodes may be utilized with the methods and devices described herein. For instance, such mapping and/or neurostimulating electrodes may be employed in conjunction with a cardiac pacemaking or defibrillation lead and more particularly a mapping and neurostimulation electrodes employed over the cardiac pacemaking or defibrillation lead while the cardiac lead is either in vivo and resident within the vascular structure. These electrodes may be placed simultaneously with neurostimulation electrodes or leads. The mapping and neurostimulation electrodes used in conjunction with the cardiac pacemaking or defibrillation lead herein is referred to as the mapping and neurostimulation electrodes.

The mapping and neurostimulation electrodes may be employed in conjunction with a cardiac lead and for interventional therapy such as neurostimulation to patients who have already had a cardiac lead installed.

Such electrodes also overcome many of the problems that exist with conventional cardiac leads or cardiac leads with integral neurostimulation electrodes. If a patient has been implanted with an existing, conventional cardiac lead and that same patient requires additional interventional neurostimulation at any point after the existing cardiac lead has been implanted, the original cardiac lead must be explanted and the entire cardiac lead must be replaced. The electrodes described herein may be installed over the excising cardiac lead and advanced down the cardiac lead body into a therapeutic position without removing or re-positioning the existing cardiac lead.

In addition, a conventional cardiac lead with integral neurostimulation electrodes, whether the neurostimulation electrodes are integral to the cardiac lead or whether the neurostimulation electrodes are sutured onto the cardiac lead, typically must be installed concurrently when the cardiac lead is originally installed into the patient. The relationship between the neurostimulation electrode and the cardiac electrode is fixed prior to implant and therefore positioning for either the neurostimulation electrodes or the cardiac electrode is sub-optimal.

Yet the electrodes described herein are completely independent and mobile and have the ability to be installed over an existing cardiac lead. Moreover, the mapping and neurostimulation electrodes can be positioned independently of the cardiac electrodes. This independent positioning ability allows for both mapping and neuro-stimulating electrodes as well as the cardiac electrode's positioning to be optimized.

The temporary or chronic mapping and neurostimulation electrodes could be inserted through several approaches including femoral, radial, right or left Subclavien veins or right or left jugular veins or in other transvenous approaches placed in veins or arteries overlapping right or left phrenic nerve. Some electrode systems/catheters could map and stimulate both phrenic nerves through transvenous approaches. In cases where there are needs for both phrenic nerves to be stimulated simultaneously, with delays, or in sequence, a single electrode/leads system or two electrode/leads systems could be deployed.

Lastly, a conventional cardiac lead with integral neurostimulation electrodes or neurostimulation electrodes sutured onto the cardiac lead body are iso-diametric and are aligned randomly. The random alignment could limit therapy because the electrical field if not focused towards the neural anatomy as the electrodes will not energize the nerve. The electrodes described herein are designed to deploy the neurostimulation electrodes and bias the neurostimulation electrode towards the vessel wall and in a position that is tangent to the neural anatomy residing outside the vessel wall. The biased or focused neurostimulation electrodes assure the electrical field induced by the neurostimulation electrodes is optimized towards the neural anatomy.

In yet another variation of a system for treating nocturnal hypoxia, the system may generally comprise one or more sensors configured to be implanted within a subject, and a controller in communication with the one or more oximetry sensors, wherein the controller may be programmed to predict a change in oxygen saturation indicative of hypoxia in the subject by identifying one or more subject-specific features from a polysomnography of the subject and correlating the one or more subject-specific features to data received from the one or more sensors, and one or more electrodes in communication with the controller, wherein the one or more electrodes are configured for placement against tissue associated with respiration of the subject and are further configured to deliver a stimulation to the tissue in response to the predicted change in oxygen saturation.

In accordance with another aspect of the invention, the one or more sensors may comprise an accelerometer, gyroscope, thoracic impedance, thoracic pressure, or heart rate sensors.

In accordance with another aspect of the invention, the system may further comprise one or more polysomnography sensors.

In accordance with another aspect of the invention, the one or more polysomnography sensors may comprise pulse oximetry, EEG, respiratory flow, ECG, or jaw movement sensors.

In accordance with another aspect of the invention, the controller may be further programmed to monitor for a fluctuation in one or more parameters including a breathing rate, tidal volume, respiratory flow, oxygen saturation, electrocardiogram, heart rate, or heart rate variability.

In accordance with another aspect of the invention, the controller may be further programmed to detect for hypoventilation or a reduced tidal volume in the subject having COPD.

In accordance with another aspect of the invention, the controller may be further programmed to provide the stimulation to the subject for initiating entrainment, breath augmentation, or lung volume increase in the subject.

In accordance with another aspect of the invention, the controller may be further programmed to detect for hypoventilation in the subject having obesity.

In accordance with another aspect of the invention, the controller may be further programmed to provide the stimulation to the subject for initiating entrainment in the subject.

In accordance with another aspect of the invention, the controller may be further programmed to detect for hypoventilation in the subject having CHF.

In accordance with another aspect of the invention, the controller may be further programmed to provide the stimulation to the subject for initiating entrainment or increasing a lung volume in the subject.

In accordance with another aspect of the invention, the controller may be further programmed to detect for an inspiration and/or exhalation profile change in the subject.

In accordance with another aspect of the invention, the controller may be further programmed to provide the stimulation to the subject for initiating a breath augmentation in the subject.

In accordance with another aspect of the invention, the controller may be further programmed to detect for a reduction in lung volume in the subject.

In accordance with another aspect of the invention, the controller may be further programmed to provide the stimulation to the subject for initiating a breath augmentation in the subject.

In yet another variation of a method for treating nocturnal hypoxia, the method may generally comprise monitoring respiration and heart rate within a phrenic nerve stimulation device implanted within a subject, predicting a potential reduction in oxygen saturation during sleep of the subject via a controller programmed to predict a change in oxygen saturation indicative of hypoxia in the subject by identifying one or more patient-specific features, and stimulating a phrenic nerve and/or diaphragm via one or more electrodes in communication with the controller for a period of time in response to the predicted change until an oxygen saturation level within the subject is normalized and hypoxia is mitigated.

In accordance with another aspect of the invention, monitoring respiration and heart rate may comprise monitoring via one or more sensors comprising an accelerometer, gyroscope, thoracic impedance, thoracic pressure, or heart rate sensors.

In accordance with another aspect of the invention, monitoring respiration and heart rate may comprise monitoring via one or more sensors comprising polysomnography sensors.

In accordance with another aspect of the invention, the one or more polysomnography sensors may comprise pulse oximetry, EEG, respiratory flow, ECG, or jaw movement sensors.

In accordance with another aspect of the invention, predicting the potential reduction in oxygen saturation may comprise predicting the potential reduction based on a fluctuation in one or more parameters including a breathing rate, tidal volume, respiratory flow, oxygen saturation, electrocardiogram, heart rate, or heart rate variability.

In accordance with another aspect of the invention, predicting the potential reduction in oxygen saturation may comprise predicting the potential reduction based on a presence of hypoventilation or a reduced tidal volume in the subject having COPD.

In accordance with another aspect of the invention, stimulating the phrenic nerve may comprise providing a stimulation to the subject for initiating entrainment, breath augmentation, or lung volume increase in the subject.

In accordance with another aspect of the invention, predicting the potential reduction in oxygen saturation may comprise predicting the potential reduction based on a presence of hypoventilation in the subject having obesity.

In accordance with another aspect of the invention, stimulating the phrenic nerve may comprise providing a stimulation to the subject for initiating entrainment in the subject.

In accordance with another aspect of the invention, predicting the potential reduction in oxygen saturation may comprise predicting the potential reduction based on a presence of hypoventilation in the subject having CHF.

In accordance with another aspect of the invention, stimulating the phrenic nerve may comprise providing a stimulation to the subject for initiating entrainment or increasing a lung volume in the subject.

In accordance with another aspect of the invention, predicting the potential reduction in oxygen saturation may comprise predicting the potential reduction based on a presence of an inspiration and/or exhalation profile change in the subject.

In accordance with another aspect of the invention, stimulating the phrenic nerve may comprise providing a stimulation to the subject for initiating a breath augmentation in the subject.

In accordance with another aspect of the invention, predicting the potential reduction in oxygen saturation may comprise predicting the potential reduction based on a presence of a reduction in lung volume in the subject.

In accordance with another aspect of the invention, stimulating the phrenic nerve may comprise providing a stimulation to the subject for initiating a breath augmentation in the subject.

These and other aspects of the invention are set forth herein in the abstract, specification and claims.

### BRIEF DESCRIPTION OF THE DRAWINGS

FIG. 1 illustrates an example of sensor use in combination with artificial intelligence (AI) to predict and/or detect nocturnal hypoxia for phrenic nerve stimulation.

FIGS. 2A, 2B and 2C illustrate respectively, flow, tidal volume and stimulation envelope signals corresponding to use of a device and method in accordance with an aspect of the invention.

FIGS. 3A, 3B, 3C and 3D illustrate respectively, EMG or another respiratory sensor within the device, flow, tidal volume and stimulation envelope signals corresponding to use of a device and method in accordance with an aspect of the invention.

FIGS. 4A, 4B, and 4C illustrate respectively, flow, tidal volume and stimulation envelope signals corresponding to use of a device and method in accordance with an aspect of the invention.

FIGS. 5A, 5B, and 5C illustrate respectively, flow, tidal volume and stimulation envelope signals corresponding to use of a device and method in accordance with an aspect of the invention.

FIG. 6 is an isometric view of a mapping electrodes mounted on a mobile sleeve which is descending over a cardiac lead;

FIG. 7 is an isometric view of a mapping electrodes mounted on a mobile sleeve which is descending over a cardiac lead and includes deployed neruo-stimulation electrodes;

FIG. 8 is an enlarged isometric view of mapping electrodes mounted on a mobile sleeve including neuro-stimulating electrodes deployed on an expandable wire member;

FIG. 9A is an exemplary side view of a neuro-stimulation electrode deployed, e.g., in a subclavian vein.

FIGS. 9B and 9C show detail side views of one mechanism for deploying the electrodes.

FIG. 10 is a detailed side view of a neuro-stimulation electrode.

FIG. 11A shows a sheath system with mapping electrodes.

FIG. 11B shows a deployed neurostimulation lead system.

FIG. 12 shows a mapping sheath and lead system with mapping electrodes inserted inside the right subclavian vein under imaging guidance.

FIG. 12 shows a neurostimulation lead loaded onto a nitinol frame/tray.

FIG. 14 shows a nitinol frame and lead system deployed inside the subclavian vein after removing the mapping sheath.

FIG. 15 shows the deployed nitinol frame lead system and implantable pulse generator (IPG).

FIG. 16 shows the collapsed nitinol frame with neurostimulation lead system.

FIG. 17 shows an expanded version of the distal end of the neurostimulation lead system including the nitinol frame and the lead.

FIG. 18 shows a symmetrical nitinol frame with prongs loaded with leads.

FIG. 19 shows an asymmetrical nitinol frame with neurostimulation leads or electrodes.

FIGS. 20A and 20B show methods for locking nitinol frame from migration and further expansion.

FIGS. 21A and 21B show examples, respectively, of the various signals measured during a sleep study for a patient with OSA and for a patient with CSA.

FIG. 22 shows a chart illustrating significant reduction of the airflow over time leading to a progressively declining level of oxygen saturation.

FIGS. 23A to 23C how examples of charts illustrating the monitoring of oxygen saturation (SpO2 or SaO2) and nasal flow during PSG for identifying desaturation events and predictive biomarkers.

FIG. 24A shows a schematic example of utilizing artificial intelligence for receiving a number of physiological inputs for generating an automated output.

FIG. 24B shows an algorithm flowchart for monitoring, prediction, and detection oxygen desaturation, hypoxia, estimated T90.

FIG. 25 shows one example of an implantable device which may include, for example, a phrenic nerve stimulation device, thoracic pressure sensing, impedance sensing, accelerometer, and gyroscope inside the device.

FIG. 26 shows another variation of a neurostimulation lead and heart rate sensor which includes one or more stimulation electrodes and one or more ECG/heart rate electrodes.

FIGS. 27A and 27B show an alternative neurostimulation lead and heart rate sensor and detail views of the sensors positioned along an expandable stent-like structure.

FIG. 28 shows another variation of a neurostimulation lead and heart rate sensor which includes one or more stimulation electrodes and one or more ECG/heart rate electrodes.

FIG. 29A illustrates an example of an implantable phrenic nerve stimulation with endovascular stimulation lead and with a thoracic pressure sensor implanted within the patient body.

FIG. 29B illustrates another example of an implantable phrenic nerve stimulation with endovascular stimulation lead with sensing capabilities incorporated directly within the stimulation device itself.

### DETAILED DESCRIPTION OF THE INVENTION

In accordance with one aspect of the invention, stimulation to elicit a diaphragm response is provided to increase or normalize lung volume and in particular to increase functional residual capacity. It is believed that stimulation to increase or to normalize lung volume or functional residual capacity may have one or more effects that may be therapeutic to cardiovascular or heart failure patients.

In accordance with this aspect of the invention stimulation may be provided using a device or method as described in one or more of the related patent applications set forth herein, to increase or normalize lung volume or functional residual capacity. For example, a bias stimulation may be provided to increase functional residual capacity or to bias lung volume for a period of time. It is believed that increasing functional residual capacity may have one or more therapeutic effects for heart failure or other cardiovascular patients, such as, for example, reducing effort required to breathe; improving gas exchange, improving SaO2 levels; providing a buffer to reduce fluctuations in blood gas levels and to reduce the likelihood of crossing the PCO2 apneic threshold; and reducing episodes of obstructive apnea in OSA patients and central sleep apnea episodes. Such buffer may also stabilize blood gases to counter fluctuations in gas levels caused by circulatory delay that may lead to Cheyne-Stokes respiration and Central Sleep Apnea. Other stimulation may be provided to achieve improved SaO2 levels or gas levels, for example, as set forth in the related patent applications which are incorporated completely and without limitation herein by reference. Other stimulation may be provided that may have the effect of normalizing lung volume, including but not limited to low frequency stimulation, low energy stimulation, or deep inspiration stimulation. These various stimulation techniques may also be provided or configured to have the effect of increasing SaO2 levels to reduce load on the heart and cardiac filling pressures.

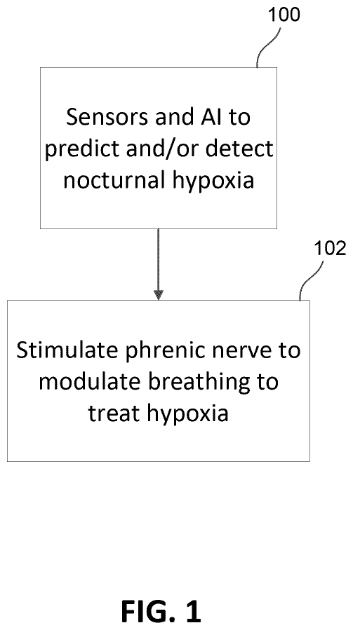

FIG. 1 illustrates one example of how sensors and algorithms incorporating artificial intelligence (AI) may be used to predict and/or detect physiological parameters associated with nocturnal hypoxia 100. One or more sensors may be located within the implantable device, or they may be integrated within or along the lead system, or the sensors in other variations may be attached to tissue regions inside or outside the body within the vicinity or attached to the patient to monitor physiological response or the phrenic nerve and/or regions of the diaphragm to receive physiological signals from the patient body. One example of the one or more sensors may include, e.g., accelerometers within the implantable device to monitor parameters such as patient breathing. In other examples, the sensors may also include electrodes, e.g., ECG and/or heart rate monitors. The one or more sensors may be attached or in electrical communication with a controller programmed to monitor and detect for any physiological signals indicative of nocturnal hypoxia. The programming may incorporate AI to automatically filter and detect for these signals in order to alert an episode of nocturnal hypoxia. If such an episode is detected, the phrenic nerve (and/or diaphragm) may be stimulated automatically by the controller to modulate breathing of the patient in order to treat or alleviate the hypoxia 102. While regions of the patient tissue such as the phrenic nerve and/or diaphragm may be stimulated, other regions of the patient body may also be stimulated in combination, separately, or alone such as tissue regions within the upper respiratory system.

In accordance with another aspect of the invention stimulation is provided to control breathing to reduce respiration rate and thereby improve, prevent or slow cardiac disease by reducing hypertension, reducing sympathetic nerve activation, providing SaO2 levels, and/or increasing cardiac output. It is believed that lowering breathing rate will provide a decrease in cardiac rate, and an enhanced vagal response.

In accordance with one aspect of the invention, breathing rate may be controlled by augmenting breathing or stimulating during intrinsic breathing to increase peak tidal volume and/or to increase inspiration duration. Increasing the duration of inspiration or tidal volume it is believed will cause the timing of the next intrinsic breath to be delayed due to the central nervous controller tendency to maintain minute ventilation in absence of any change at the chemoreceptor level. The rate may be continuously slowed by detecting each intrinsic breath and providing stimulation or augmenting until the duration of inspiration, tidal volume or exhalation rate is at a level that brings the breathing rate to a desired rate which is reduced by the central nervous control of minute ventilation.

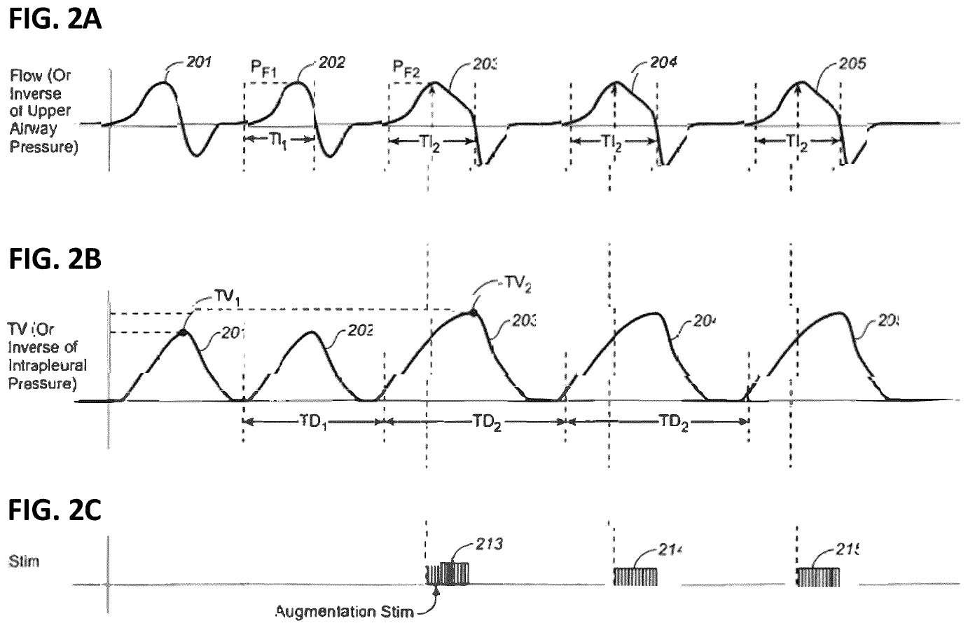

FIGS. 2A to 2C illustrate stimulation during intrinsic breathing in accordance with one aspect of the invention. FIG. 2A illustrates flow for breaths 201, 202, 203, 204 and 205. FIG. 2B illustrates tidal volume of breaths 201, 202, 203, 204, and 205. Breaths 201, 202 are intrinsic breaths. Breaths 203, 204, and 205 are intrinsic breaths that are augmented by stimulation configured to elicit a diaphragm response as illustrated schematically by stimulation markers 213, 214, and 215.

Stimulation is initiated at a period of time during inspiration and is provided for a period a time in a manner configured to normalize or increase tidal volume. Stimulation during intrinsic breathing and augmenting breathing are described in one or more related applications as set forth herein which are incorporated completely and without limitation herein by reference. The tidal volume TV2 of the breaths 203, 204, 205 where inspiration is augmented is greater than the tidal volume TV1 of the intrinsic breaths 201, 202. According to one variation, the peak flow during stimulation Pf2 may be configured as shown to be close to the peak flow Pf1 during intrinsic breathing. The inspiration duration TI1 of intrinsic breathing is shorter than the inspiration duration TI2 of augmented breaths 203, 204, 205. The duration TD1 of intrinsic breathing is increased to duration TD2 and with stimulation signals 213 214, 215, to achieve a desired rate.

In accordance with another aspect of the invention, stimulation during intrinsic breathing may be provided to inhibit or delay onset of next inspiration. According to an aspect, stimulation may be provided during exhalation to inhibit or delay onset of an inspiration thereby slowing breathing rate. According to an aspect, stimulation may be provided to extend exhalation thereby delaying the onset of a subsequent inspiration. According to an aspect, stimulation may be provided at a low energy, low level or low frequency to inhibit onset of an inspiration, thereby slowing breathing rate. Examples of low energy, low level and/or low frequency stimulation are set forth in the related applications herein. Very low level stimulation may be also used to maintain the phrenic nerve in an activated mode in order to communicate with the brain or central respiratory center in the brain regarding phrenic nerve livelihood. This neuromodulation model may not elicit any or any significant diaphragmatic response leading to changes in lung or tidal volume but may be used as a communication path with the central respiratory drive to stay awake or at such active levels to maintain normal breathing mechanism. Examples of these types of neurostimulation may include, e.g., sacral nerve stimulation for treatment of urinary incontinence or bowl disease. Sacral nerve stimulation activates passages to the brain to maintain normalcy of urinary function. These neuromodulation modalities do not necessarily cause a muscle response.

The rate of intrinsic breathing may be controlled by delaying intrinsic breaths with low energy (for example a lower amplitude, frequency and/or pulse width than desired for paced breathing) diaphragm stimulation provided during intrinsic breathing.

According to one aspect, low energy stimulation may be provided during intrinsic breathing, delaying onset of the next breath and thereby slowing breathing rate. According to another aspect, stimulation may be initiated sufficiently prior to the onset of the next breath so as to reduce the likelihood that the stimulation would trigger a breath. A combination of lower energy stimulation and timing the stimulation sufficiently prior to the onset of the next breath may be used to slow breathing rate.

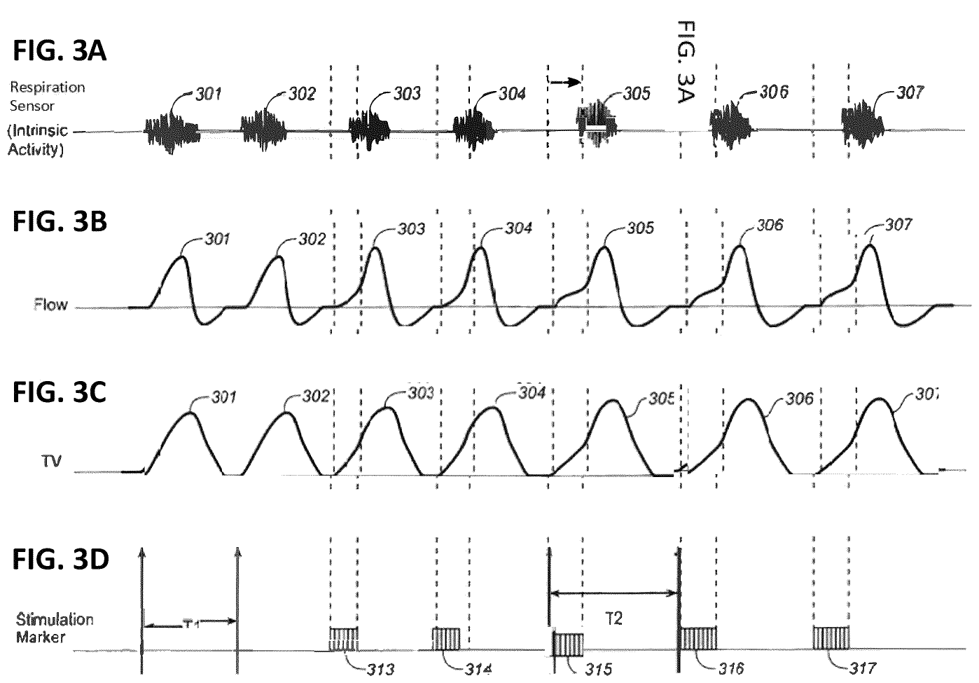

FIGS. 3A to 3D illustrate stimulation provided to slow breathing in accordance with one aspect of the invention. FIG. 3A illustrates intrinsic diaphragm respiration activity detected by a respiration sensor corresponding to breaths 301 through 307. FIGS. 3B and 3C respectively illustrate flow and tidal volume corresponding to breaths 301 through 307. FIG. 3D illustrates stimulation envelopes corresponding to stimulation signals 313, 314, 315, 316, and 317 provided prior to onset of breaths 303, 304, 305, 306, and 307 respectively. Stimulation 313, 314, 315, 316, 317 is provided prior to the onset of breath 303, 304, 305, 306, 307 respectively, as determined, for example, by a model that predicts the onset of breathing or by the actual detection of the intrinsic diaphragm EMG activity (FIG. 3A). Stimulation is sustained for a period of time. For example, the stimulation may be provided until the onset of the intrinsic breath is detected by the EMG or other physiological signals. As illustrated, the stimulation increases the duration of a respiration cycle T2 with respect to the duration T1 of an intrinsic breathing cycle. As further illustrated, intrinsic breathing cycles 303 to 307 may have greater flow or tidal volume to compensate for the slower breathing rate that is induced by the stimulation.

In accordance with another aspect of the invention, stimulation to increase tidal volume or inspiration duration may be provided in combination with stimulation during exhalation to inhibit or delay the onset of the next inspiration.

In accordance with another aspect of the invention stimulation may be provided to delay exhalation by stimulating at the end of inspiration at a level that slows exhalation. Such stimulation may be provided by stimulating during intrinsic breathing or by providing paced breathing for example that maintains minute ventilation while providing a slower rate of breathing.

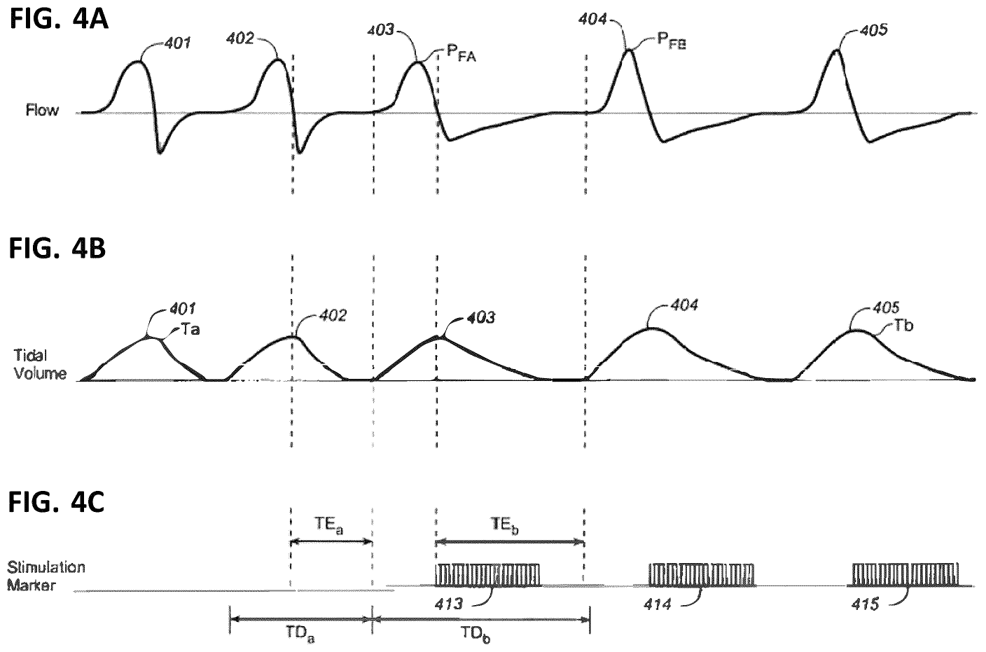

FIGS. 4A-4C illustrate stimulation during intrinsic breathing in accordance with one aspect of the invention. FIG. 4A illustrates flow for breaths 401, 402, 403, 404 and 405. FIG. 4B illustrates tidal volume of breaths 401, 402, 403, 404 and 405. Breaths 401, 402 are intrinsic breaths. Breaths 403, 404, and 405 are intrinsic breaths that are augmented by stimulation configured to elicit a diaphragm response as illustrated schematically by stimulation markers 413, 414, and 415. Stimulation is initiated at a period of time at the end of inspiration and is provided for a period a time through the exhalation period. Detection and stimulation techniques are set forth, for example in related applications hereto. Stimulation may be provided at a low energy level including at a low frequency. Stimulation during intrinsic breathing and augmenting breathing, low level and/or low frequency are described in one or more related applications as set forth herein which are incorporated completely and without limitation herein by reference. The peak flow during stimulation Pfb may be greater than the peak flow Pfa during intrinsic breaths 401, 402 as illustrated. The peak flow during stimulation Pfb may be also not be greater than the peak flow Pfa during intrinsic breaths 401, 402. Similarly tidal volume Tb is for breaths 404, 405 after stimulation 413 and 414 respectively. Such greater flow or tidal volume may intrinsically compensate for the slower breathing rate that is induced by the stimulation. It is believed that stimulation during exhalation inhibits or delays onset of inspiration. The stimulation also slows exhalation (i.e., during the period which exhalation is occurring at a relatively faster rate) so that the exhalation duration TEb during stimulation is greater than the intrinsic exhalation duration TEa. Exhalation is slowed by stimulation thus slowing the overall rate of breathing. The duration of the intrinsic breathing respiration cycle TDa is increased to duration TDb during stimulation, thus reducing the breathing rate to a desired rate.

Stimulation may also be provided to slow or control breathing rate in a manner that provides a paced breath with controlled exhalation as illustrated for example in U.S. patent application Ser. No. 10/966,474, filed Oct. 15, 2004 and U.S. patent application Ser. No. 10/966,472, filed on Oct. 15, 2004.

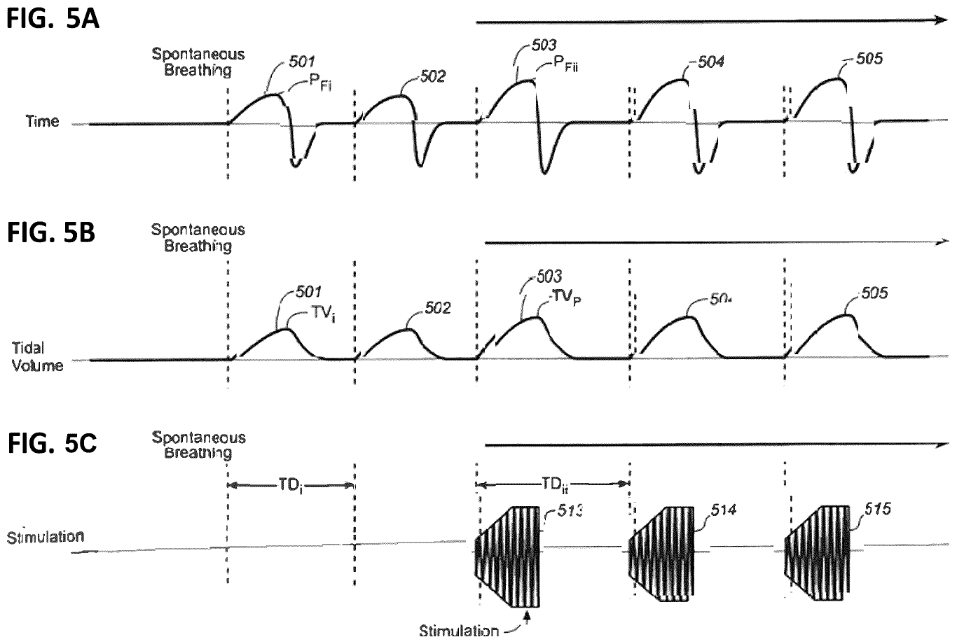

FIGS. 5A to 5C illustrate stimulation used to control breathing and breathing rate in accordance with the invention. Breaths 501 and 502 are intrinsic breaths occurring at a rate such that the duration of the respiration cycle is TDi and having tidal volume TVi and peak flow PFi. Breaths 503, 504 and 505 are paced breaths with higher tidal volume TVp and peak flow PFp. Peak flow PFp may be controlled to be at a level substantially the same as, higher, or lower than intrinsic peak flow. Paced breathing is provided in a manner in which breathing is controlled or taken over by stimulated breathing. Examples of techniques for controlling breathing, respiratory drive and/or taking over breathing are set forth in related applications incorporated completely and without limitation herein by reference. In general greater tidal volume permits a reduction in breathing rate or an increase in duration of breathing cycle to TDii while maintaining minute ventilation. FIG. 5C illustrates stimulation envelopes 513, 514, 515 respectively corresponding to stimulated breaths 503, 504, 505.

In accordance with another aspect of the invention stimulation is provided to control minute ventilation to therapeutically affect blood gas levels. Examples of controlling minute ventilation are set forth for example in U.S. patent application Ser. No. 10/966,474. Such stimulation may be provided, for example, during sleep to thereby increase or normalize SaO2 levels during sleep. In accordance with one aspect of the invention minute ventilation is controlled to normalize SaO2 levels while not decreasing PaCO2 levels close to the apneic threshold. According to this aspect minute ventilation may be actively controlled using sensors to sense SaO2 and hypoxia and//or PaCO2 levels which may be used as input parameters for a controller utilizing AI in determining the stimulation to be applied. Weaning off of pacing may be desirable to insure that the intrinsic drive to breath is still present. Paced breathing may be calibrated, for example at implant or adjusted during device use, so that the device is able to provide the appropriate minute ventilation at each pacing setting. This information may be obtained for example through sleep studies where the device is designed to provide stimulation during sleep.

In accordance with another aspect of the invention, stimulation is provided to create a deep inspiration or an increased tidal volume to thereby reduce sympathetic nerve bias, improve blood gas levels, stimulate reflexes (for example the Hering-Bruer reflex related to activating stretch receptors), increase lung volume, normalize or reset breathing (one or more parameters) or provide other beneficial therapies to improve cardiovascular function or heart failure condition.

Examples of creating deep inspiration are set forth in U.S. patent application Ser. No. 11/272,353 filed Nov. 10, 2005. While these examples refer to using deep inspiration to treat apnea, similar techniques for stimulation may be used to create deep inspiration breaths for improving cardiovascular function or treating heart failure. Stimulation may be provided during intrinsic inspiration or in between inspiration cycles.

In accordance with another aspect of the invention stimulation may be provided to manipulate intrathoracic pressure to thereby produce a therapeutic effect.

According to one embodiment, stimulation is provided to reduce intrathoracic pressure through induced contraction of the right and/or left hemidiaphragm. It is believed that for some patients, reduction in intrathoracic pressure may have a beneficial effect on the patient's cardiovascular function or condition. For example, a reduced intrathoracic pressure may increase stroke volume at least in part through a decrease in central venous pressure; and reduce pulmonary arterial and wedge pressures in relation to atmospheric. A reduced intrathoracic pressure may also provide a decrease in filling pressure in the right ventricle and may also thereby improve systemic venous return. A reduced intrathoracic pressure may also provide better coronary artery perfusion.

In accordance with one aspect of the invention, patients with heart failure manifesting in poor ventricular filling may be treated with stimulation to reduce intrathoracic pressure. In accordance with one aspect of the invention, patients with diastolic heart failure may be treated with stimulation to reduce intrathoracic pressure. In accordance with another aspect of the invention stimulation to reduce intrathoracic pressure may be provided to patients who are hypovolemic where the therapeutic effects of improved ventricular filling and venous return would be particularly beneficial.

According to one aspect of the invention stimulation is provided to elicit a diaphragm response to cause a reduced intrathoracic pressure. The stimulation is provided at a level that does not elicit a breath, in other words, where intrinsic breathing continues to occur. Examples of stimulation such as bias stimulation and low energy or low frequency stimulation are described in related applications set forth herein. The stimulation eliciting a reduced intrathoracic pressure may be sustained or intermittent. Stimulation is preferably provided when a patient is sleeping but may also be provided when a patient is awake.

In accordance with one aspect of the invention, stimulation may be provided to one hemidiaphragm to elicit a more impactful change in intrathoracic pressure in the respective side of the thoracic cavity. For example the right hemidiaphragm may be stimulated in such a way to cause a reduced intrathoracic pressure primarily in the right thoracic cavity to thereby effect the right side of the heart to a greater degree than the left. Or stimulating unilaterally on the diaphragm may serve to minimize the pressure changes that the heart is exposed to. This may be beneficial when an increased lung volume is desired to treat OSA or CSA. Sensors may be used to sense arterial and venous blood volume so that stimulation may be adjusted based on patient's blood volume state. For example, stimulation may be increased or turned on when the patient is in a hypovolemic state where in a particular patient a greater benefit would be produced with a more negative intrathoracic pressure. Such sensors may include, for example, impedance (plethysmography) sensors used to monitor fluid levels in the body. Separate electrodes, or existing stimulation electrodes may be used in a configuration or with frequencies that can determine resistance and/or reactance. Fluid volume changes may, for example, be monitored based on a baseline established with the sensors and a hyper or hypo volemic state may be detected. A list of possible sensors are described in the references above.

In accordance with another aspect of the invention, stimulation is provided to elicit a diaphragm response that improves heart failure as described above in combination with treating sleep disorders that contribute to or worsen heart failure. Accordingly, stimulation is provided as described in the related patent applications set forth herein, to elicit a diaphragm response to thereby reduce breathing disorders to thereby improve condition of a heart failure patient. One or more specific methods of reducing sleep disordered breathing events and preventing sleep disordered breathing are described in related applications as set forth herein. In accordance with one aspect of the invention, stimulation is provided prior to a physiological trigger of a central or obstructive sleep apnea event in a manner that reduces the occurrence of such events, thus reducing the effects of apnea events that worsen heart failure.

In accordance with another aspect of the invention a combined cardiac rhythm management device and diaphragm/phrenic nerve stimulation device is provided to provide an enhanced combined treatment device. In accordance with this aspect of the invention, the diaphragm stimulation element may comprise an abdominally placed stimulator positioned on the diaphragm or phrenic nerve, a thoracoscopically placed stimulator positioned on the diaphragm or phrenic nerve, a phrenic nerve stimulator positioned in the neck region on or adjacent the phrenic nerve (transcutaneous, percutaneous, or otherwise implanted); transcutaneous stimulation of the diaphragm through leads at or near the ziphoid region (this may be in combination with a defibrillator function or device that is configured for subcutaneous stimulation of the heart); or a pectorally positioned lead, for example, placed transvenously in a vein or artery in proximity of one or both phrenic nerves . . .

The system may be further enhanced through the ability to avoid negative device/device interactions where a separate controller is used, e.g. for a CRT, pacemaker, ICD or other therapeutic electrical stimulation device. The system may also provide arrhythmia and sleep disorder detection algorithms through sensing of both the cardiac and respiration cycles.

The system may also be included in a combination with a cardiac rhythm management (CRM) device having a common controller.

Additionally, the system may also be utilized to provide a continuous or synchronized low level stimulation to the phrenic nerve or diaphragm overlapped with the patient's own intrinsic breathing to reduce an intrathoracic pressure and improve cardiac output. The patient's SaO2 levels may also be improved and the heart and respiration rates may be reduced.

This may be achieved in part by sensing and/or monitoring the patient's SaO2 levels and applying the continuous or synchronized stimulation, as described herein, to stabilize SaO2 within the nominal or normal range for that patient. In applying the stimulation to the patient's phrenic nerve or diaphragm, any of the sensing and stimulation devices and methods described in the following may be utilized for applying the continuous or synchronized low level stimulation: U.S. Patent Application Ser. Nos. 61/893,404 filed Oct. 21, 2013; 60/925,024 filed Apr. 18, 2007; Ser. No. 13/598,284 filed Aug. 29, 2012; Ser. No. 12/082,057 filed Apr. 8, 2008; Ser. No. 12/082,057 filed Apr. 8, 2008; Ser. No. 12/069,823 filed Feb. 13, 2008; Ser. No. 12/044,932 filed Dec. 21, 2007; Ser. No. 11/981,342 filed Oct. 31, 2007; Ser. No. 11/480,074 filed Jun. 29, 2006; Ser. No. 11/271,315 filed Nov. 10, 2005; Ser. No. 11/271,554 filed Nov. 10, 2005; Ser. No. 11/271,353 filed Nov. 10, 2005; Ser. No. 11/271,264 filed Nov. 10, 2005; Ser. No. 11/480,074 filed Jun. 29, 2006; Ser. No. 11/271,726 filed Nov. 10, 2005; Ser. No. 10/966,487 filed Oct. 15, 2004; Ser. No. 10/966,484 filed Oct. 15, 2004; Ser. No. 10/966,474, filed Oct. 15, 2004; Ser. No. 10/966,421 filed Oct. 15, 2004; Ser. No. 10/966,472 filed Oct. 15, 2004; Ser. No. 10/686,891 filed Oct. 15, 2003. Each of these applications is incorporated completely and without limitation herein by reference for any purpose.

Another application of this device/technology is to improve cardiac hemodynamics by increasing venous return and cardiac output.

Another application of this technology includes applying any phrenic nerve stimulation mention in this application or applying negative pressure therapy even in the absence of changes in hemodynamic or SaO2 to improve oxygenation and cardiac output and off-loading the heart. In the long-term, the heart could remodel and improve contractility on its own.

The implantable sensor could receive energy from outside the body such as the CardioMEMS pulmonary pressure sensor and then receive commands to stimulation phrenic nerve to reduce pressures and increase cardiac output. The stimulation electrodes could be also activated from outside the body.

Such devices could also synchronize its stimulation of the phrenic nerve to cardiac cycles such systole or diastole. However, in order to achieve sustained reduction in pulmonary or atrial pressures, a sustained stimulation that is synchronized to respiration cycles and also cardiac cycles may be provided. Intrathoracic pressure is lowest at the peak of inspiration and therefore while it is possible to stimulate, the stimulation applied toward the end of inspiration and/or part of or the entire exhalation phase may be more efficient.

The stimulation algorithm could be targeted toward multiple benefits/targets. At the time of device implant, the algorithms for each target could be titrated and thresholds could be established per patient:
1. Proactive stimulation during sleep or awake to increase cardiac output in diastolic or systolic heart failure patients;
   a. Device will self-adjust stimulation relative to the need for certain cardiac output increase;
2. Responsive therapy where the device monitors pressures or cardiac and intrathoracic impedances or cardiac output and therefore responds to need to reduce intrathoracic pressure;
3. Responsive device to increase cardiac output;
4. Responsive device to increase lung volume;
5. Integrated with any CRM device; pacemaker, defibrillator, cardiac resynchronization therapy (CRT);
6. Integrated with other heart failure devices such as vagal nerve stimulation or others;
7. Integrated with sleep apnea therapy devices including hypoglossal nerve stimulation devices.
8. Responsive therapy device to improve kidney function or improve GFR
9. Responsive device to reduce pulmonary pressures and pulmonary congestions
10. Responsive device to prevent hypoxia
11. Proactive device to maintain oxygen saturation within normal boundaries of the patient

In one example, because the algorithms for each target are able to be titrated, the phrenic nerve or diaphragm tissue may be stimulated to cause a titratable diaphragm contraction such that an initial pressure within a thoracic chamber is reduced. In another example, the phrenic nerve or diaphragm tissue may be stimulated to improve a cardiac output in titratable manner as well.

In stimulating the phrenic nerve or diaphragm as well as monitoring the patient's intrathoracic pressure, as described herein, the electrodes may be utilized in combination with or integral to a cardiac lead. Such electrodes are described in further detail in U.S. Patent Application Ser. No. 61/893,404 filed Oct. 21, 2013, which has been incorporated by reference hereinabove in its entirety and for any purpose.

The mapping and neurostimulation electrodes presented herein are intended to be used in conjunction with or integral to a cardiac lead. They could also be an independent lead. The mapping electrodes mounted on the sleeve is intended to traverse the cardiac lead, provide specificity to specific neural activation points within the vascular structure where neural anatomy resides adjacent to the vascular structure, such as the phrenic or vagus nerve. Once the targeted nerve anatomy is identified by the mapping electrodes, the neurostimulation electrodes can be arranged or deployed within the vascular structure and adjacent to the neural anatomy such that the electrodes provide the desired neurostimulation therapy.

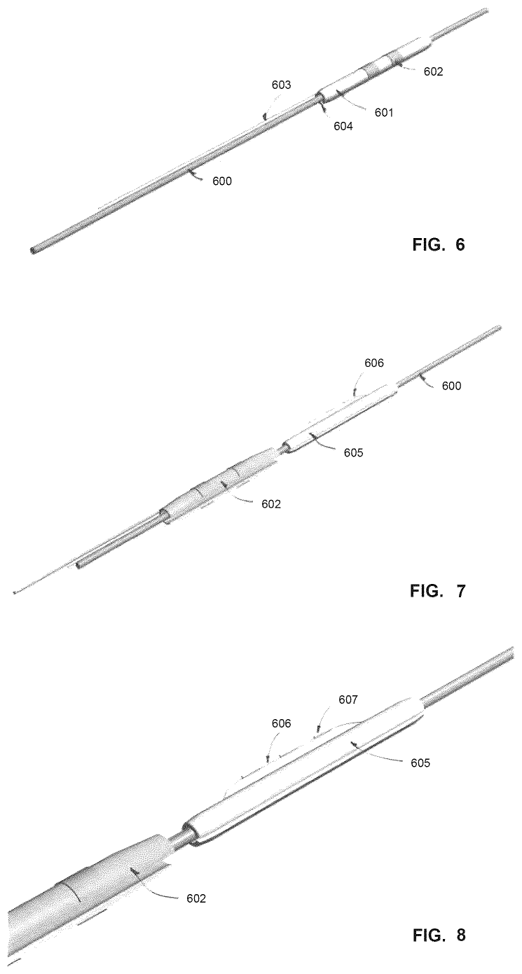

FIG. 6 illustrates an embodiment of a mapping sleeve 601 that includes at minimum one but in this embodiment plural mapping electrodes 602, traversing a cardiac lead 600. The mapping sleeve 601 in this embodiment is inserted over the cardiac lead 600 at the proximal end of the lead and advanced along the cardiac lead body to a position in which the mapping electrodes 602 are arranged to activate neural anatomy.

The mapping sleeve 601 may be constructed of a bio-stable polymer, silicone rubber, or other insulation materials suitable for isolating a plurality of electrodes. The mapping electrodes 602 may be constructed of platinum or platinum alloys but in other embodiments constructed of any bio-stable conductor, titanium, palladium, stainless steel, carbon, or similar materials, alloys. or composite materials.

Once the neural anatomy is identified within the vascular structure, the mapping sleeve 601 is retracted as illustrated in FIG. 7 exposing an inner sleeve 605 that includes an expanding wire member 606. The wire member may be constructed of any bio-stable compliant metal, nitinol, stainless steel, titanium alloys, or plastic material suitable to expand into position.

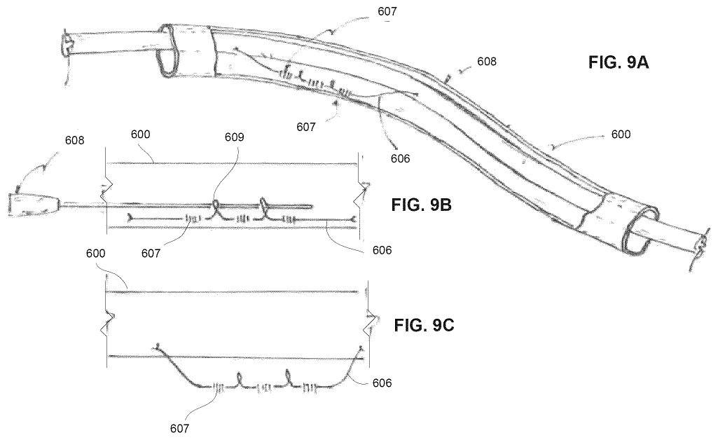

As illustrated in FIG. 8, the expanding wire member 606 in which carries at least one but in the preferred embodiment, plural neurostimulating electrodes 607. The expanding wire member 606 when in the un-deployed state, resides under the mapping sleeve so that the entire assembly can negotiate the vascular structure. The wire 606 may be retained in its low-profile configuration through various mechanism, such as a stylet 608 which may be passed through one or more retaining loops 609 defined along the wire 606, as shown in the detail view of FIG. 9B. When deployed, the stylet 608 may be retracted such that the expanding wire member 606 expands, as shown in the detail view of FIG. 9C, to apply the neurostimulation electrodes 607 against the vascular wall, as shown in FIG. 9A. In this example, the lead 600 may utilize a IS-1 type connector.

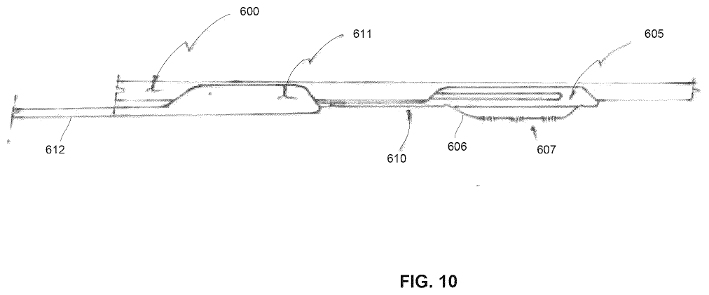

FIG. 10 illustrates an embodiment of the deployed neurostimulation electrodes 607 expanded to reside coincident to the vessel wall 608. In the primary embodiment, the electrode wire 606 containing the neurostimulation electrodes 607 have expanded to focus the electrodes 607 current towards the neural anatomy residing outside the vascular structure. In this variation, the electrode wire 606 and electrodes 607 may be attached or coupled to a conductor cable 610. A push sleeve 611 may be slidingly positioned proximally or distally of the electrode 607 with a proximal end of the push sleeve 611 being coupled to a push rod 612. During lead insertion and intravascular delivery, the pushing sleeve 611 may remain over the wire 606 and electrodes 607. When the electrodes 607 are in position relative to the tissue wall, the push rod 612 may be actuated proximally or distally relative to the lead 600 such that the push sleeve 611 is moved to expose the wire 606 and electrodes 607 which may then be deployed as the sleeve 611 is, e.g., retracted.

The mapping electrode may be advanced down a previously implanted cardiac lead body to a point in which neural structure intersects the vascular structure. The mapping electrode is used to identity “map” the optimal stimulation location or optimal location to place the neurostimulation electrodes within the vascular structure.

Once the optimal stimulation location is identified using the mapping electrodes, the neurostimulation electrodes are deployed such that the neurostimulation electrodes are positioned in a location to energize the targeted neural anatomy.

A method of mapping or identifying the nerve is developed where once the electrode is near proximity of the nerve, stimulations of variety of frequencies and amplitude will be applied in certain sequence for optimum nerve location. The physiological response to mapping procedure will be monitored and recorded. Once the electrode is in optimum location, the electrode location in reference to other anatomical landmarks are noted and the electrode is secured. In case of mapping the phrenic nerve, several physiological parameters including diaphragm movement and response, flow, tidal volume, lung volume, minute ventilation, upper airway muscle activity, and similar parameters as it relates to respiratory parameters will be monitored in order to identify the optimum electrode placement in reference to the phrenic nerve.

A neurostimulation lead system may be comprised of a commercial lead mounted and connected to a flexible frame fabricated from nitinol or other biocompatible material where the frame may be inserted and advanced via flexible catheter body through a vessel into proximity of a nerve to be stimulated. The frame can be constructed of nitinol wires where the nitinol wires act as a “guidewire” which can be inserted through a lumen. Alternatively, the lead can be placed and fastened to the frame such that as the frame expands, the lead may adhere to the vessel wall in the intended position where the lead is facing the targeted nerve through the vessel. The frame and lead system may be housed or inserted inside a sheath or mapping sheath. Specific markers and labels on the sheath and sheath catheter may provide orientation of the lead and electrodes in reference to the targeted nerve. As the sheath is pulled away (e.g., pulled proximally), the nitinol frame may slowly expand in the intended location within the vessel and orientation relative to the nerve to be stimulated. The frame/lead system can also be collapsed by pushing the sheath distally over the frame and repositioned and released again and/or removed from the vessel.

The lead system including the frame/holder/tray may be constructed in such a way to hold more than one neurostimulation leads in symmetrical or asymmetrical positions. The frame's distal end could be one or multiple nitinol wires apposing to the vessel wall connected to each other or extending open like a regular stent. It is preferred to allow blood flow in the vessel as least interrupted as possible. In one variation, if the nitinol wires are connected to each other at the distal end, it is preferred they will be oriented against the vessel wall and not in the middle of the vessel. If the nitinol wires are not connected to each other or the tray, they should be apposed against the vessel wall in such a way to minimize vessel wall injury or perforation. The vein walls are highly complaint while the blood flow is of lower velocity. Design considerations should take into account to minimize vessel wall injury and perforation. The leads may have, e.g., 2-4 electrode contacts, where two are selected for threshold and nerve activation purposes. If two leads each with, e.g., three electrodes, are placed asymmetrically on the side of the vessel wall in proximity to the nerve to be stimulated, this configuration may allow for the selection of the best electrode pairs from both leads or just one lead. In case of slight lead migration, endothelization, or changes to lead-tissue interface impedance overtime, another set of electrodes can be selected for optimum performance. There is a possibility more than one electrode is configured as a single pole to increase the area of stimulation, for example, targeting the nerve with three electrodes instead of two and increasing the area of stimulation.

With the electrode deployed within the vessel and oriented to stimulate an adjacent nerve or nerves of interest, a treatment may be initiated by applying stimulation through the electrodes which may pass through the vessel wall and into the targeted nerve or nerves. Parameters including current, power, pulse width, timing, etc. may be varied for treating various conditions. Various treatments to be applied may include electrical stimulation for treating or mitigating conditions such as breathing disorders and/or sleep-related breathing disorders where treatment parameters which may be implemented by the electrode systems described herein are described in further detail in the following references, each of which are incorporated herein by reference in its entirety: U.S. Pat. Nos. 8,467,876; 8,255,056; 8,412,331; 8,200,336; 8,348,941; 7,970,475; 9,259,573; 8,140,164; 8,116,872; 9,370,657; 8,265,759; 10,857,363. Other examples of treatments which may be applied via the electrode systems described herein are described in further detail in the following references, each of which are incorporated herein by reference in its entirety: U.S. Pat. Nos. 8,244,358; 7,979,128; 8,160,711; 8,335,567; 8,280,513; and U.S. Pub. 2021/0060342A1.

In one variation of the electrode system, the leads may be removed after chronic implantation within the body of the patient. The nitinol frame may be coated with a biocompatible material such as silicone to prevent endothelialization. The frame may be fabricated from a biocompatible polymer such as polyether ether ketone (PEEK) and optionally coated with a material such as an anti-thrombolytic (e.g., HemoLAST™, AST Products, Inc., MA). The entire frame and lead may be collapsed and removed after a period of time such as up to a year. A position of the lead may be maintained by designing the spring force of the nitinol to be low enough to expand against the vessel wall but not create enough force to erode its way through the tissue. The nitinol frame could be made of one diameter nitinol tube and be fitted into variety of vessel diameters or be made of variety of nitinol tube diameters and during the procedure with the aid of imaging, e.g., ultrasound imaging, the optimal size and fit lead system can be selected and deployed after the vessel diameter is measured with the aid of imaging, such as ultrasound.

In another variation, the lead may be non-removable after chronic implantation. In this case, the nitinol frame may be endothelialized within the vessel wall after a period of time, such as thirty days or longer.

Whether the leads are removable or intended to remain within the patient, cither version of the leads may be removable acutely and up to, e.g., thirty days of implantation. Furthermore, the implantation and/or treatment procedure may allow for the addition of adjunct procedures utilizing devices such as catheters, interventional devices, pacer leads, etc.

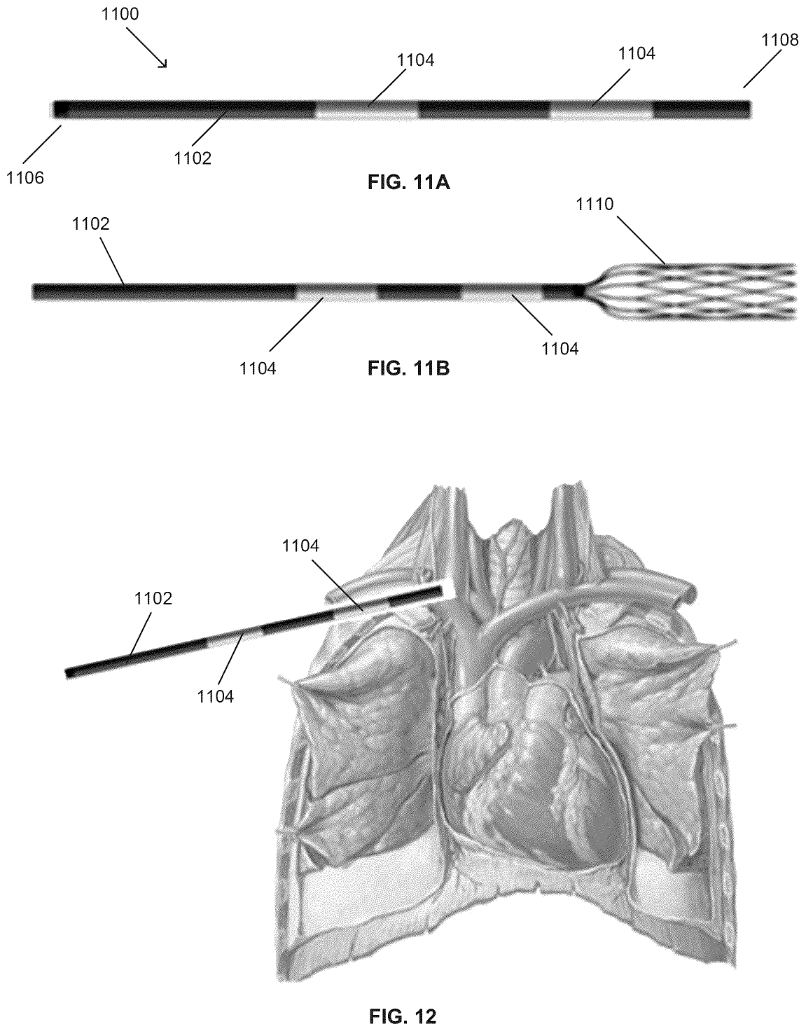

FIG. 11A shows a side view of an endovascular lead apparatus 1100 having an elongated body with a mapping sheath 1102 or catheter having one or more active electrodes 1104 placed thereon as discrete electrodes separated from one another along a length of the mapping sheath 1102. While two electrodes 1104 are illustrated in FIG. 11A, other variations can incorporate a single electrode or more than two electrodes in other configurations. The electrodes 1104 can be spaced apart at varying distances, as described in more detail below.

The apparatus 1100 may be introduced and advanced through the patient body intravascularly to position the apparatus into proximity of the nerve to be treated. Upon advancement and delivery to a target site, the electrodes 1104 can be actuated to deliver a treatment stimulation to the target site via an energy source. The treatment stimulation can be used to confirm a location of the nerve body to be treated in proximity to the vessel. The sheath 1102 can also be slowly moved towards the proximal portion 1106, thus allowing an expandable scaffold which may deploy from a low-profile delivery configuration to an expanded deployment configuration into contact against the inner walls of the vessel. One example is shown illustrating a nitinol frame 1110 coupled to an inner member and extending in its deployed configuration from a distal opening in the distal portion 1108 of the sheath 1102, as shown in FIG. 11B. The expandable scaffold may be entail any number of deployable scaffolds so long as the scaffold is deliverable in a low-profile and then deployed in an expanded configuration into contact against the interior vessel walls.

As shown in one example in FIG. 12, the sheath 1102 can be inserted into the subclavian vein and advanced intravascularly until the electrodes 1104 are positioned into proximity to, e.g., the phrenic nerve which crosses behind the subclavian vein. To confirm the treatment location, a pulse generator located externally of the patient or implanted within the patient body may be actuated such that the electrical stimulation is delivered from the controller and through the length of the sheath 1102 and to the electrodes 1104 along the mapping sheath 1102 such that the electrical stimulation passes through the vessel wall and into the phrenic nerve. When nerve capture is detected through observation or measurement of the patient's diaphragm contraction or a respiratory parameter, the electrical stimulation may be halted.

FIG. 13 shows another variation of an expandable scaffold in nitinol frame 1110 which may be deployed from a mapping sheath 1102 or catheter. In this embodiment, a tray 1300 or holder positioned along a second side of the distal portion opposite to the first side can be provided to position a frame 1110 such as a nitinol frame. The electrodes 1104 may be positioned along the first side of the sheath 1102. The tray 1300 can be placed on one side of the apparatus 1100 and can comprise a channel 1302 in which the frame 1110 can sit in a low-profile delivery configuration. When the frame 1110 is ready to be deployed within the vessel, an expansion mechanism coupled to a proximal end of the one or more frame members can be actuated to transition the frame into an expanded, deployed configuration, as seen in FIG. 13. The sheath 1102 can flexibly bend in the expanded configuration while the frame 1110 may extend from the second side and also press against an interior of the vessel wall and urge the first side of the distal portion 1108 and the electrodes 1104 into contact against an inner surface of the vessel.

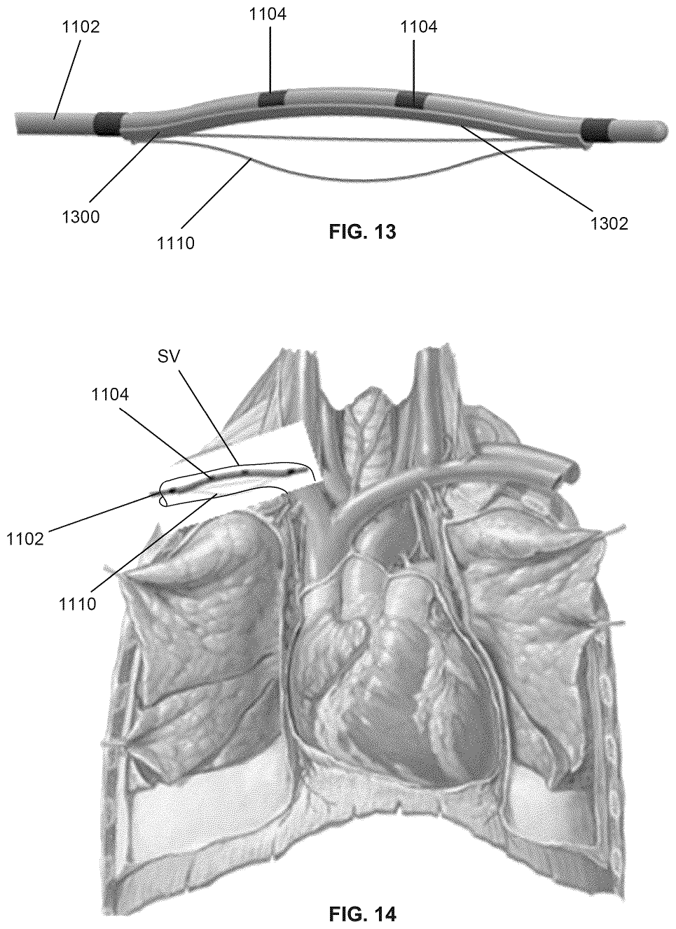

FIG. 14 shows an example where the frame 1110 and endovascular lead apparatus 1100 may be deployed inside the subclavian vein SV after removing the mapping sheath 1102. Alternatively, the lead system can be loaded onto a wire instead of a frame or tray 1300. The wire can be made of nitinol or another suitable material (e.g., PEEK, etc.).

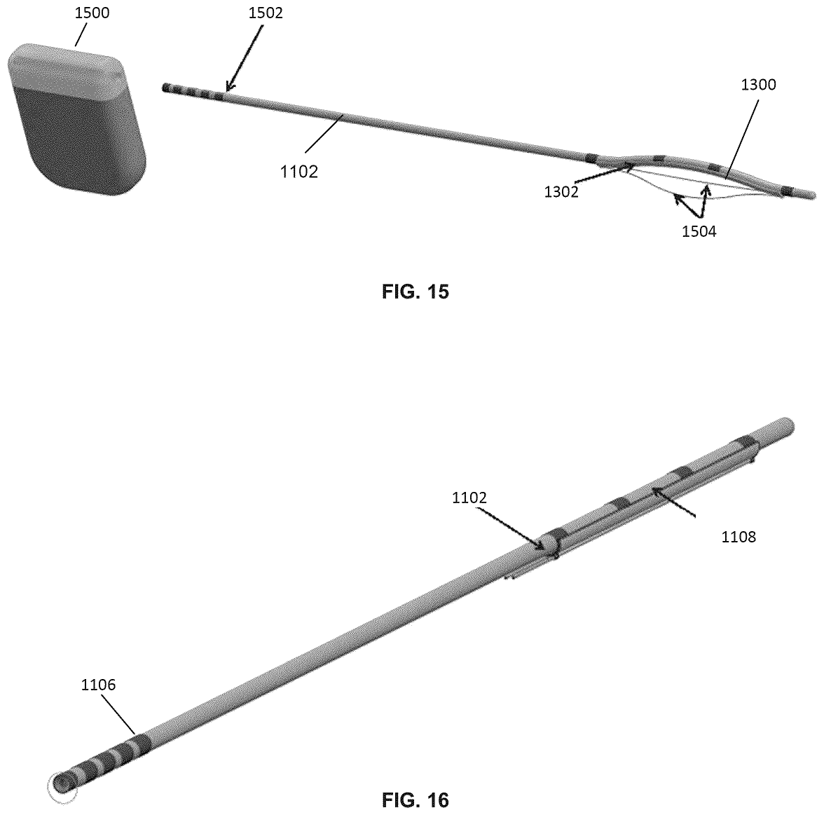

FIG. 15 illustrates the endovascular lead apparatus 1100 attached or attachable to an implantable pulse generator (IPG) 1500 which may be implanted within the patient body or which may be located external to the patient body. The IPG 1500 may be electrically coupled to the mapping sheath 1102 through one or more electrical connectors or contact leads so that the IPG 1500 is electrically coupled to the electrodes located at the distal end of the device. As shown, the IPG 1500 can be electrically coupled to the proximal portion 1106 of the sheath 1102 at one or more contact leads 1502 positioned at a proximal end of the sheath 1102 and can deliver energy from the proximal portion 1106 of the apparatus 1100 to the distal portion 1108 of the sheath 1100. In some variations, the IPG 1500 can comprise a transducer for delivering electrical energy to the electrodes using an energy source such as ultrasound. The electrical energy generated can have a frequency between, e.g., 1 Hz and 400 Hz. For example, a frequency of an electrical impulse can be set at a relatively low frequency (between about 1 Hz to 10 Hz), a medium frequency (between about 10 Hz to 150 Hz), and a high frequency (between about 150 Hz to 400 Hz). In another variation, the lead system can be loaded onto one or more wires 1504 instead of a frame or tray 1300. The one or more wires 1504 can be made of nitinol or another suitable material (e.g., PEEK, etc.).

Alternatively, the wires within a lumen of the catheter 1102 can include the one or more wires 1504 used to urge the electrodes against the interior of the vessel wall. The neurostimulation lead apparatus thus can be constructed with a hollow lumen or channel 1302 defined through the sheath 1102 where the nitinol wire 1504 can be inserted. Upon removal of the mapping sheath 1102, the nitinol wires 1504 may be expanded (either self-expanding or expanded via actuation) to the intended diameter allowing the neurostimulation lead to be placed against the vessel wall in proximity to the nerve to be treated.

FIG. 16 shows a perspective view of the neurostimulation lead apparatus 1100 and the nitinol frame 1110 in its collapsed configuration. In one variation, the outer diameter of the sheath 1102 can be, e.g., about 7 Fr. The lead can include one or more electrodes 1104, e.g., 2-8 electrodes, where the electrodes may be spaced apart from, e.g., about 2 mm to about 5 mm. The electrodes 1104 can be equally separated from each other. Alternatively, the electrode separation can be arbitrary, uniform, or non-uniform. For example, the distal electrodes 1104 can be, e.g., 5 mm from one another, while proximal electrodes 1104 can be, e.g., 2 mm, from one another. There can be multiple configurations of electrode separation in one lead.

The electrodes 1104 can be made of various conductive materials, e.g., gold, iridium, palladium, a gold-palladium-rhodium alloy, rhodium, etc., or a combination thereof. In some embodiments, the electrodes 1104 can be made of a metallic composite with a high charge injection capacity (e.g., a platinum-iridium alloy or composite).

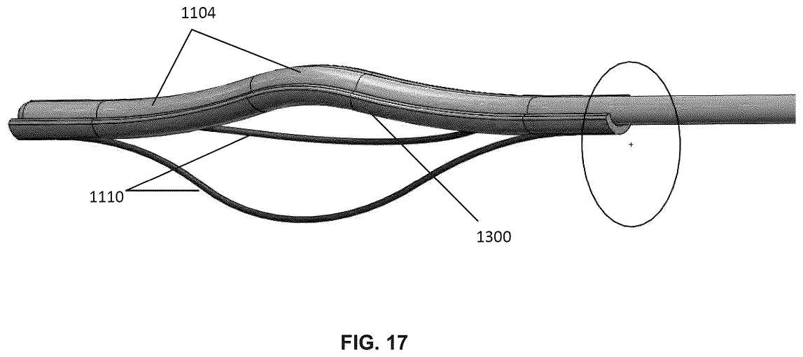

FIG. 17 shows a perspective detail view of another variation of the distal end of the neurostimulation lead including the nitinol frame 1110 and electrodes 1104. The tray 1300 and frame 1110 can be attached along a side of the frame 1110 when the frame 1110 is in the deployed configuration. The nitinol frame 1110 can be reconfigured into a non-linear shape and can abut the inner walls of the vessel to urge the tray 1300 and electrodes 1104 into contact with the opposite portion of the inner wall into proximity to the phrenic nerve for treatment.

The device can be used in another locations within a body, including but not limited to: the carotid artery, the vagus nerve, the cervical sympathetic ganglion, the aorta, the vagus nerve, the superior mesenteric ganglion, the inferior mesenteric ganglion, the renal artery, the renal nerves, the subclavian artery, the brachial plexus, the common hepatic artery, the gastroduodenal artery, the iliac artery, and the splanchnic nerves.

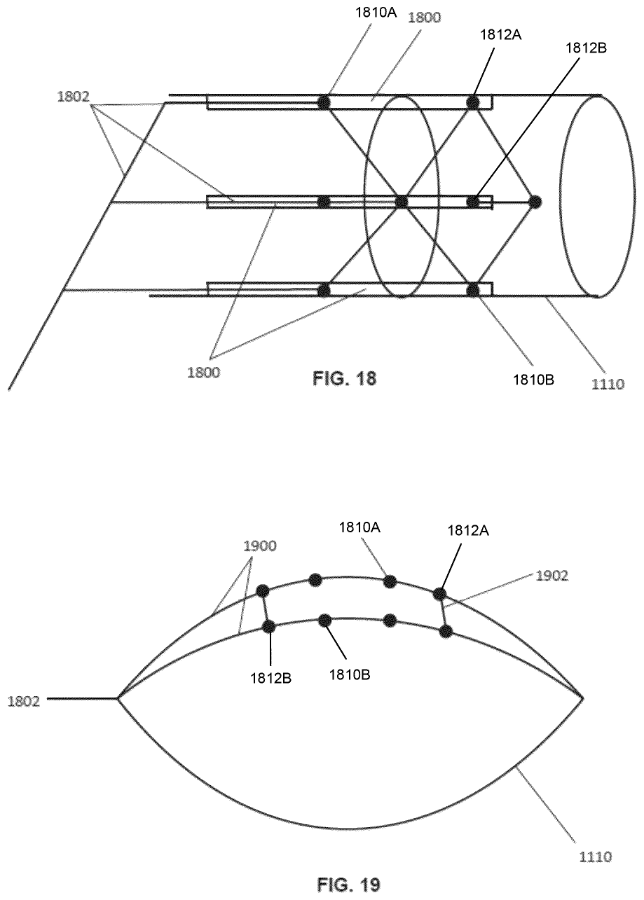

FIG. 18 shows a symmetrical nitinol frame 1110 having multiple prongs 1800 (e.g., anywhere from two, three, four, five, etc.) which may deploy into an extended scaffold-like structure with each of the prongs 1800 having one or more stimulation electrodes 1104. This configuration allows for multiple electrodes configurations to identify most optimum electrodes (e.g., pairs or higher numbers) and optimum threshold settings between at least two of the prongs 1800. For instance, two or three prongs 1800 may be used to apply the stimulation in a cross-cross configuration.

The prongs 1800 can be coupled to the frame 1110 and can comprise wires 1802 or leads extending thereon which can couple to a stimulation electrode pair such as stimulation electrodes 1810A, 1810B which may be placed at different areas on the frame 1110. In this example, the stimulation electrodes 1810A, 1810B may be positioned on opposite sides of the frame 1110. Furthermore, while the stimulation electrodes 1810A, 1810B are shown as a pair of electrodes, multiple pairs may be incorporated or an uneven number of stimulation electrodes may also be used. Alternatively, the stimulation electrodes may be positioned at various locations along the frame 1110 so that the electrodes are not positioned opposite to one another. The frame 1110 can be shaped to expand and collapse with the electrodes 1810A, 1810B and wires 1802 maintaining their positions relative to each other. The variation in FIG. 18 shows a cross-cross stimulation selection, though it should be understood that other shapes and configurations can be designed (e.g., circular, elliptical, etc.). Electrodes 1810A, 1810B can be placed on frame 1110 through any number of attachment mechanisms such as by welding or crimping a conductive insulated wire assembly directly onto the frame 1110 and/or each individual electrode.

The frame 1110 may also incorporate one or more electrodes which are used to detect other physiological parameters such as ECG and/or heartrate related signals. An example is illustrated where frame 1110 may incorporate one or more ECG/heartrate electrodes 1812A, 1812B which may be positioned at various locations along frame 1110.

The frame can contain stress and strain in strategic locations of the scaffold where diametric contraction or expansion is translated as relative angular displacement of struts. Diametric contraction or expansion, for example, can occur when the frame is crimped, expanded at deployment, or over-expanded at deployment. The frame can contain the stress and strain in the peaks and valleys of the frame and the angular arrangement of the struts is designed to allow a finite amount of over-expansion beyond the original intended diameter. Any expansion beyond the predetermined level may cause strut elongation, reduced radial force, increased diametric recoil, and decreased structural integrity and fatigue life. Solution casting a frame having such design characteristics provides another way of optimizing the frame.

Wires 1802 can be made in part of any number of conductive materials, e.g., platinum tungsten, gold, aluminum, nitinol wire, rhodium, iridium, nickel, nickel-chromium alloy, gold-palladium-rhodium alloy, chromium-nickel-molybdenum alloy, and/or stainless steel, etc. In addition to nitinol, the frame 1110 can be made at least in part of, e.g., stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof. The frame can also be made in part of a shape memory polymer.

FIG. 19 shows yet another variation of an asymmetrical nitinol frame 1110 having neurostimulation leads and stimulation electrodes 1810A, 1810B concentrated along a select portion of the frame so that the stimulation electrodes 1810A, 1810B along the frame may be directed towards or near the nerve to be stimulated when the frame 1110 is expanded from a low profile delivery configuration to an expanded deployment configuration. One example may utilize four poles (while other variations may utilize less than four or greater than four poles) where the nitinol wires 1900 are asymmetric from one another and can have, e.g., about 2 mm to about 3 mm, distance between the wires 1900. Likewise, one or more ECG/heartrate electrodes 1812A, 1812B which may be positioned at various locations along select portions of the frame 1110 such that the ECG/heartrate electrodes 1812A, 1812B are deployed into contact against the particular tissue regions of interest for measurement. One or more stent links 1902 which may extend between adjacent longitudinal scaffold members can be utilized between the wires to provide for stability during expansion or deployment.

While the nitinol frame 1110 may be released from a constrained, delivery configuration to an unconstrained, expanded configuration when released, a number of various methods and devices may be implemented to control the expansion of the nitinol frame to appose to the vessel wall in a manner similar to stent expansion and the frames may also be locked into place to minimize expansion.

In any of the variations described where multiple members of the nitinol frame 1110 are implemented, each of the individual members may be coupled with one another at a common connection point such as the distal and/or proximal ends of the frame. However, in other variations, each of the individual members may be arranged independently from one another rather than having a common connection point. In yet other variations, the individual members may be connected to one another at a proximal or distal end while the opposing end remains independent of one another.

Yet another variation may utilize one or more of the members of the frame being connected to the elongate device at a single end. In one example, the proximal ends of the frame members, e.g., three members, may be attached at their proximal ends to the elongate device such that the members upon expansion may open or flare into a configuration where the distal ends of the members extend away from elongate device.

In yet another variation, the leads may be integrated directly into the nitinol frame rather than having the leads separately located along the distal portion of the elongate device. In this manner, the frame may reconfigure into its expanded configuration into contact against the tissue walls where the leads may directly contact the tissue as well via the frame. Treatment may be implemented by electrical stimulation being delivered through the frame members instead.

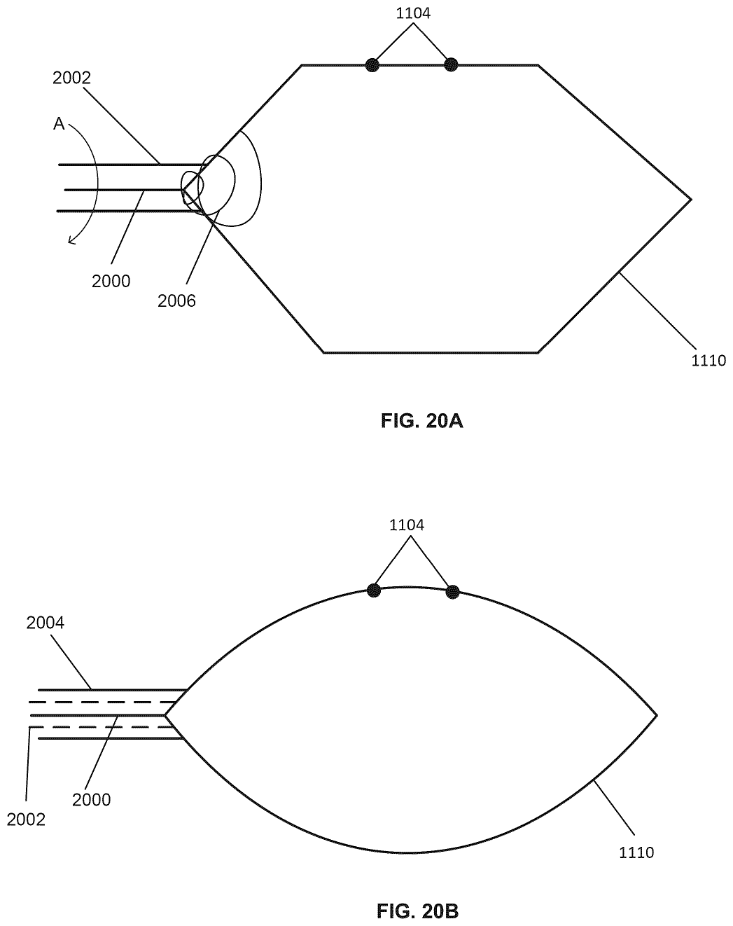

Given that vessel walls are compliant, the frame 1110 can be constrained to prevent any inadvertent nitinol expansion over time that may cause vessel wall injury or perforation. In one variation, FIG. 20A illustrates a side view of a rotatable tether mechanism 2006 attached to an end, such as the proximal end, of the nitinol frame 1110. A flexible shaft 2002 can be positioned through the length of the elongate sheath and the flexible shaft may be rotatable about its longitudinal axis, as seen by the arrow A in FIG. 20A. A distal end of the shaft may incorporate the rotatable tether mechanism 2006 such as a helical member which encompasses the proximal ends of the reconfigurable frame 1110. Rotation of the helical member in a first direction may expand the frame into its expanded configuration and rotation in a second opposite direction may collapse the frame back to its low-profile configuration. The controlled expansion of the nitinol/PEEK or other type material frame may be deployed into contact against the vessel wall (e.g., with the aid of imaging) in a controlled expansion.

Once the desired expansion is reached, the shaft 2002 of the tethering mechanism can be locked to thereby prevent or minimize the nitinol frame 1110 from further expansion. The expansion of the nitinol frame 1110 through the locking mechanism can be adjusted in such a way that the user can determine how many millimeters the nitinol frame 1110 has expanded. Markers and the number of rotations can guide the user to expand the frame to a desired diameter. This locking mechanism attached to the neurostimulation lead may also prevent the frame migration when the lead is attached to the IPG 1500.

FIG. 20B shows a side view of yet another variation for controlling the expansion or collapse of the nitinol frame 1110. A push-pull sleeve 2004 or shaft can be slidably positioned over the length of the elongate shaft 2002 (shown in dashed lines for clarity) such that the distal end of the push-pull sleeve 2004 is slidably connected to a proximal portion or end of the nitinol frame. Once the frame 1110 is ready for expansion, the push-pull sleeve 2004 can be pulled proximally to allow for the expansion of the frame to appose against the vessel. A position of the push-pull sleeve 2004 may be locked relative to the lead body outside of the vessel to prevent the further expansion of the frame 1110. The body can be twisted, terminated, and removed from the lead body while the frame 1110 is locked in the position. Alternatively, the push-pull sleeve 2004 can be part of the lead body and in case of lead repositioning or removal, it can un-lock the frame 1110 and collapse it by winding it down for removal from the vessel.

An alternative mechanism incorporates a flexible tether 2000 coupled to a distal portion or end of the frame 1110. As pulling of the tether 2000 may tension it due to the reconfiguration of the frame from its deployed configuration to its expanded configuration, maintaining tension upon the tether 2000 may maintain the expanded configuration of the frame. A securement mechanism, such as a friction lock, on a proximal portion of the tensioning tether may lock the tether 2000 in place. The securement mechanism may be configured to automatically release, e.g., by a retrieval catheter or a cutting mechanism which may cut the tether 2000.

In ensuring a sufficient level of oxygen saturation in a patient during sleep, an algorithm may be used to predict the reduction in oxygen saturation during sleep prior to an apnea event or due to a cardiovascular, obesity, and/or pulmonary function. An implantable device alone may be implemented within a subject or an implantable device in combination with external sensors and/or devices may be utilized. The device may use phrenic nerve stimulation to treat the potential reduction in oxygen saturation and therefore mitigate apnea and minimize hypoxia thereby minimizing cardiovascular morbidity and mortality associated with hypoxia.

Accordingly, one objective is for the diagnostic prediction and detection of oxygen saturation, oxygen desaturation, and T90 monitoring trends. Using the sensors within the device and system and applying machine learning techniques at the patient level from polysomnography data and expert labeling including the oxygen desaturation levels and the time spent under 90% (T90), the system may predict and detect when a patient may progress toward a significant drop in oxygen saturation level for a period of time leading to arousals or high T90 episodes. Changes in oxygen saturation could be because of possible SDB (desaturation) or non-specific desaturation due to poor circulation, heart failure, AFIB, COPD, obesity, or a combination of factors.

It is assumed each patient has an arousal SaO2 threshold meaning when SaO2 drops below that range and it leads to hypoxia and possibly an arousal takes place. While this concept may vary from patient to patient, the algorithm (using polysomnography data) can specify a stable or normal SaO2 level which is the result of certain breathing and cardiovascular characteristics. When the breathing and cardiovascular characteristics change such that it leads to unstable SaO2 and hypoxia and further to a level causing arousal. Hence, respiration and/or heart rate may be monitored with implantable sensors to predict and detect when a change in breathing and/or heart rate could result in a reduction in SaO2 possibly leading to hypoxia and possibly arousal. The changes in respiration may be modeled using polysomnography data and AI and changes in patterns of an accelerometer output in the device.

### Polysomnography Analysis

Each patient diagnosed with sleep apnea can undergo one or multiple polysomnography (PSG) evaluations. The first PSG is generally for diagnostic purposes which could be at home or sleep laboratory. Many of the FDA approved PSG systems monitor SaO2 in addition to other parameters so a profile of SaO2 can be made available from the very early diagnostic process. Assuming the patient is diagnosed with moderate to severe sleep apnea needing some therapy, the next PSG may be conducted at a sleep lab when the appropriate CPAP pressure is titrated.

PSGs at home or sleep labs may include a finger pulse oximetry sensor and other sensors such as flow, heart rate (HR), EEG, and optionally others. A PSG can be analyzed, annotated, and scored by an expert showing, for example, apnea, hypopnea, arousals, changes in SaO2, heart rate, as well as other parameters or combinations of these parameters. The polysomnography may be scored by conventional devices or processes as well as being reviewed by an expert to confirm the results.

The prediction and detection algorithm utilizes multiple factors for each patient including, for example: frequency of apneas and hypopneas, associated SaO2 and changes in SaO2 from normal to apneic threshold, progression of SaO2 as reference to arousal threshold, calculation of T90, validation of T90_{desat} VS T90ₙₛ, associated breathing patterns, average heart rate (HR), changes in HRV, and potentially other parameters to strengthen the oxygen desaturation progression prediction algorithm. In addition, the algorithm may continuously, on programmed intervals, or based on event trigger can be programmed to predict oxygen saturation levels and then calculate the T90 levels whether caused by SDB or non-specific contributors. One objective of the prediction algorithm is to use patterns in respiration including breathing rate, slope of inspiration and exhalation, delays to next inspiration (rest period), tidal volume, minute ventilation, heart rate, HRV, and chest movement to predict an event leading to reduction in SaO2 and SaO2 arousal threshold. Other parameters such as presence of lung disease such COPD and asthma, systolic and diastolic heart failure, AFIB, CAD, and other morbidities may be included in data collection and possibly contributing to predictive algorithm. Another objective is to utilize phrenic nerve stimulation to maintain SaO2 above arousal threshold and T90 value, as discussed in further detail herein.

It is validated that many apnea and cardiovascular patients generally have normal awake ventilation, minute ventilation (MV), and SaO2 (e.g., MV=5 L/min and Sa02-96%). However, during sleep and due to multiple reasons, both ventilation and SaO2 will drop to either a new lower steady state normal levels (e.g., MV=3.5 L/min and Sa02=89-90%) or abnormal levels leading to significant reduction in SaO2 causing hypoxia, arousals, associated morbidities, impact on quality of life, and cardiovascular stress. The causes of reduction in SaO2 to abnormal levels include, for example: SDB, airway narrowing and obstruction, reduction in respiratory drive (central), hypoventilation, reduction in lung volume due to obesity or other reasons, reduction in cardiac output, and COPD.

Additional inputs to the algorithm may include sleeping position (e.g., supine, right side, left side), REM and NREM sleep stages as they could contribute to accuracy of the predictive model/algorithm.

A new steady state normal MV and SaO2 versus abnormal levels as a cause for arousals and hypoxia could vary from patient to patient or it could be patient-specific. Thus, a patient-centric approach of developing a machine learning algorithm engine based on each patient PSG and additional data can be implemented. The algorithm (“PSG-algorithm”) may identify feature selections or principal components from the PSG and additional data to predict an onset of reduction in SaO2 and arousals.

The PSG-algorithm may then be overlapped and correlated with the device sensors to develop a “Device-Algorithm”. The Device-Algorithm may use breathing and/or heart rate sensors from within the device (e.g., common sensors between PSG and device and possibly bedside or wearable external sensors) to predict progression toward reduction in SaO2 before a respiratory event (e.g., apnea, hypopnea, CSA, Cheyne-Stoke, airway narrowing, reduction in lung volume) or an arousal event takes place.

In one embodiment, the device sensor can include a 3D accelerometer and/or gyroscope that can measure chest movements and patient position and correlate to or predict an SDB or other biomarkers leading to reduction in oxygen saturation, hypoxia, and T90s. This may include changes in tidal volumes or slopes of inspiration and exhalation, rate of change in inspiration or exhalation, or changes in minute ventilation. Each patient's “normal range” of MV, breathing rate, tidal volume, and other respiratory and heart parameters can be logged in the algorithm baseline as “normal”. Deviations from “normal” parameters can be analyzed and enable the device to decide which mode of phrenic nerve stimulation (therapy) is suitable to overcome the deviation and prevent reduction in oxygen saturation, hypoxia, and associated respiratory, sleep, and cardiovascular consequences.

The algorithm can target certain sensitivity and specificity. However, the algorithm can minimize false negatives for each patient and thereby minimize hypoxia. While the algorithm intends to minimize false positives, the false positives could lead to additional therapies which are safe while false negatives lead to not delivering therapies when patients need it the most and therefore exposing patients to additional cardiovascular risks.

In predicting and detecting T90_{desat}, breathing patterns and principle components from the predictive algorithm (described herein) may be identified and time measurements used to predict and detect T90_{desat}.

In predicting and detecting T90ₙₛ, since T90ₙₛ takes place due to hemodynamic factures such as cardiac output, pulmonary hypertension, COPD, obesity, and/or the hypoventilation syndrome, in addition to respiratory sensors data T90ₙₛ may be predicted with HR and HRV sensor data. The machine learning algorithm may identify principle components and contributors toT90ₙₛ from the polysomnography data and overlapped and/or correlated to implantable sensors.

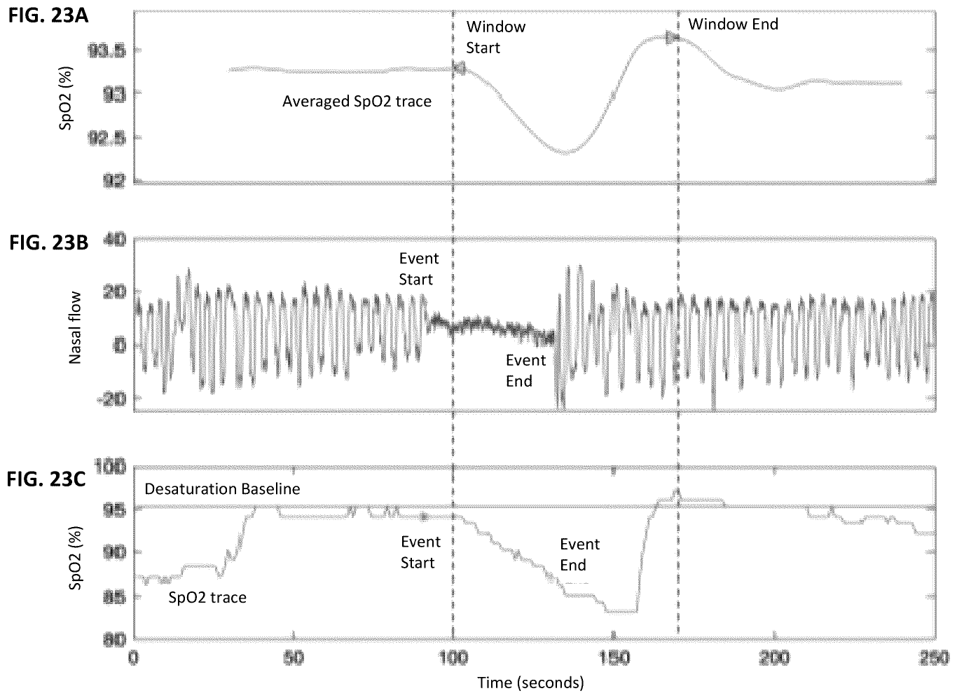

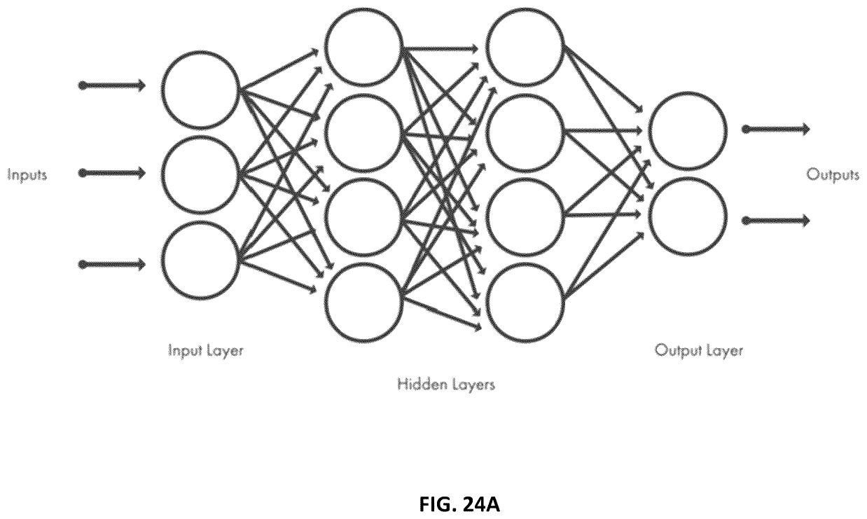

FIGS. 23A to 23C show examples of charts illustrating the monitoring of oxygen saturation (SpO2 or SaO2) and nasal flow during PSG for identifying desaturation events and predictive biomarkers.

In one example, each patient PSG, a normal range of MV and SaO2 or baseline may be established. This could include, for example, patient sleeping position, sleep stages, certain tidal volume, respiratory rate, minute ventilation, inspiration and exhalation slopes, and associated HR, and HRV are analyzed. Additional parameters such as patient disease morphology (e.g., CAD, AFIB, COPD, Heart failure, Stroke) could be included as stated above. The delay in SaO2 sensors may be accounted for in the algorithm development. Overlapping or common features between PSG and implantable sensors may be identified. Since sleep-related breathing disorder (SBD) is progressive and therefore predictive, the progress or transitions to hypopnea, apnea, flow limitation, Cheyne-Stoke, CSA, or mixed apnea can be all categorized. Correlating continuous pulse oximetry with the above sensors data, a d_SaO2 predictive and machine and deep learning models may be created. In correlating the implantable sensors and characteristics with the above PSG sensors and data, one can develop a continuous d_SaO2. This model can be optimized like regenerative AI models. A d_SaO2 sensor can be utilized to calculate the T90 and initiate therapies when T90 or d_SaO2 fall below a certain threshold based on the programmed parameters for the specific patient.

The d_SaO2 digital sensor and algorithm can be calibrated using, for example, wearable oxygen saturation or sleep apnea devices and applications (e.g., as used on smart devices) with oxygen saturation sensors to further optimize the algorithm on a periodic basis, e.g., monthly or quarterly basis. Such wearable products & technologies are currently available on the market, for example, Sunrise Health (Sunrise SA, Namur, Belgium) wearable device or Itamar (Itamar Medical, Inc., Atlanta, Georgia) at home devices. There are many products on the market providing information that can support algorithm optimization. The data from these wearables can be uploaded to a cloud and used for optimizing the prediction and detection algorithm and then downloaded to the implantable device.

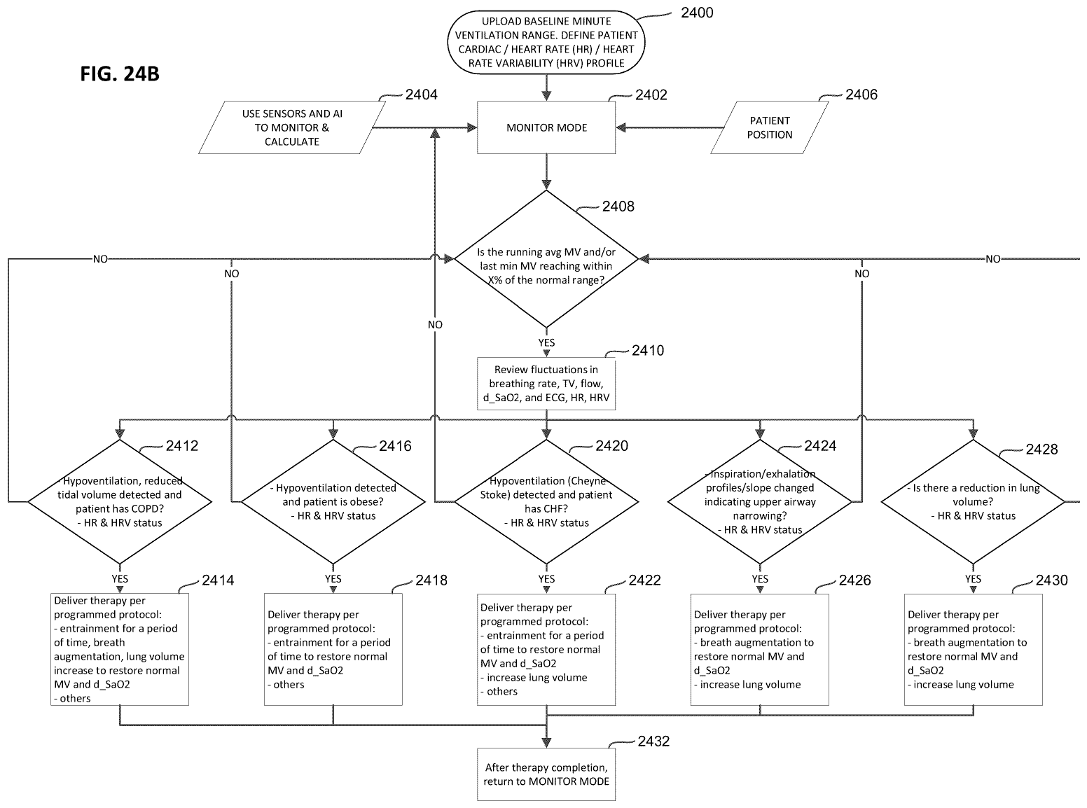

Another approach to predicting changes in SaO2 during sleep and monitoring and predicting T90_{desat} and T90ₙₛ is the utilization of Deep Learning as illustrated schematically in FIG. 24A. The “Inputs” are from a polysomnography or a wearable variation of a device that may monitor one or more specified parameters for each patient including, e.g., sleep stage, sleep position, time of the night, breathing pattern, SaO2, heart rate, heart rate variability, EEGs, chest movement, tidal volume, minute ventilation, arousals, apnea, hypopnea, flow limitations, CAD, AFIB, COPD, etc., and other sensors may be fed as inputs to the Input Layers. Any number of these Inputs and/or any number of combinations of these Inputs may be monitored. The Output or Outputs may include, e.g., SaO2 along with other output parameters, d_SaO2, SaO2 alone, and/or predictive models of hypoxia, etc. Any one or more combinations of these Outputs may be used to derive therapy and/or phrenic nerve stimulation output levels. The deep learning modeling may take the multiple Inputs and model the Output to represent one or more sensors such as an accelerometer and/or heart rate sensor, etc. Outputs could be more than just one parameter and may include the accelerometer (e.g., representing chest movement), SaO2 level, probability of T90_{desat} and/or T90ₙₛ, even the need for therapy and what type of therapy should be delivered to treat the specific changes to SaO2.

The deep learning model may be used to predict or detect hypoxia based on stored models or patterns (e.g., Hidden Layers) for the specific patient. This AI model can identify when a hypoxia or even modest reduction in SaO2 can take place and inform the system or the output to prepare for therapy deliveries. The model may use, e.g., breathing, cardiovascular, chest movement, patient position, and other parameters to develop and update the model. The deep learning model may receive new data from the same patient from within the implantable device, bedside or external devices, and polysomnography data to optimize the model over the life of the patient. This model may create a single sensor pattern to inform the device/system when to deliver what type of therapy.

For example, the sensor could be a 3D accelerometer and deep learning model can train the algorithm based on the output of the accelerometer alone. The accelerometer output patterns could model the outcome of the polysomnography or a wearable sleep monitoring device. In this manner, the device could have one breathing sensor. In addition, the heart rate and heart rate variability sensors could be trained with polysomnography deep learning models and act as a single sensor. In order to improve sensing and detection accuracy, the system could use dual or more deep learning models. An example is training both the accelerometer and heart rate sensors simultaneously or independently to mimic polysomnography outcomes.

Another approach and relevant to measuring/calculating T90 may be accomplished using a real-time oxygen saturation digital sensor (d_SaO2) within the device based on a respiratory sensor variations such as accelerometer, thoracic impedance, thoracic pressure, HR, and HRV. The d_SaO2 sensor and algorithm may correlate each patient's polysomnography data (including pulse oximetry) with respiratory, HR, and HRV sensors.

An algorithm flowchart for monitoring, prediction, and detection oxygen desaturation, hypoxia, estimated T90 is illustrated in FIG. 24B. Generally, once the program is awake and sleep patient's stable MV, TV, RR, HR, inspiration and exhalation characteristics leading to upper airway and lung compliance characteristics (use prior PSG and data from implantable and external sensors accompanied by the device), these parameters from a first PSG may be validated or reprogrammed with the implantable sensors (e.g., sleep lab or home device). The internal sensors may be calibrated to the PSG sensors (e.g., accelerometer movement correlated to flow and TV from PSG). Using PSG and device sensors data, a stable range for MV, TV, RR, respiratory signal characteristics, HR, and HRV may be defined where an objection is maintaining at stable SaO2 range. The PSG and implantable sensor data may be used to calculate e-T90 (estimated T90) where a variation in MV may correlate to SpO2. The various parameters such as minute ventilation range, patient cardiac values such as HR and/or HRV may be used to create a patient profile which is uploaded 2400, e.g., to the controller.

The algorithm to monitor MV, TV, RR, respiratory signal characteristics, HR, and HRV, and may flag/detect any of the parameters related to SaO2 which are outside of a 80% or 90% “SaO2 stable range” where primary parameters are MV & HRV and where HRV changes may be due to a reduction in MV in cardiac patients.

When the algorithm detects SaO2 is progressing outside SaO2 stable range, the algorithm may characterize an event as respiratory (desaturation) or non-specific such as cardiac, obesity, or COPD related. For example, if desaturation is respiratory related, identify reduction in MV due to reduction in flow, TV, RR, Cheyne-Stoke, CSA, or other respiratory issues. If desaturation is not respiratory related (is non-specific), label desaturation as non-specific. Once the root cause is identified as one of the above, go to therapy module. After completion of therapy, the algorithm may go to monitor mode.

Generally, the flowchart of FIG. 24B describes hypoxia prediction, potential causes, and specific prevention protocols (therapy algorithm). It starts with uploading a baseline minute ventilation range:
During wakefulness at rest
During sleep in supine position
During sleep in left side or right side position
During sleep in REM & NREM sleep states
Patient clinical characteristics: CHF, Obese, CAD, MI

The algorithm begins with a MONITOR MODE 2402 for monitoring various parameters using use internal and/or external sensors and AI (as described) to monitor 2404, for example, RR, calculate TV, and d_SaO2 as well as patient position 2406, as shown:
Monitor minute ventilation (MV) during sleep
Calculate running average of MV.
Compare running average of MV to the last minute MV
Monitor heart rate and calculate HRV
Calculate respiration rate (RR), tidal volume (TV), d_SaO2

The algorithm then determines if the running average MV and/or last minute MV reaches within a predetermined percentage (X %) of the normal range 2408. If so, then fluctuations in breathing rate, TV, flow, d_SaO2, ECG, HR, HRV are reviewed 2410. If the MV is outside a predetermined stable range (setup by algorithm for that patient and calibrated to device sensors), the detection algorithm looks underneath for TV, flow, respiration rate, changes in inspiration/exhalation slope leading to airway narrowing, and then diagnose if hypopnea, apnea, CS, CSA, reduction in LV, etc. could be the cause of changes in MV. Thus, the algorithm decides on therapy algorithm to stabilize MV and SaO2 in order to prevent or mitigate both forms of hypoxia (SDB and non-specific related).

In reviewing the fluctuations 2412, the algorithm may determine, for example, is hypoventilation, reduced tidal volume detected and patient has COPD? The HR and HRV status may also be reviewed. If so, then deliver therapy 2414 per programmed protocol:
Entrainment for a period of time, breath augmentation, lung volume increase to restore normal MV and d_SaO2
Other therapies

In reviewing the fluctuations 2416, the algorithm may alternatively determine, for example, is hypoventilation detected and patient is obese? The HR and HRV status may also be reviewed. If so, then deliver therapy 2418 per programmed protocol:
Entrainment for a period of time to restore normal MV and d_SaO2
Other therapies

In reviewing the fluctuations 2420, the algorithm may alternatively determine, for example, is hyperventilation (Cheyne-Stoke) detected and patient has CHF? The HR and HRV status may also be reviewed. If so, then deliver therapy 2422 per programmed protocol:
Entrainment for a period of time to restore normal MV and d_SaO2
Increase lung volume
Other therapies

In reviewing the fluctuations 2424, the algorithm may alternatively determine, for example, are inspiration and exhalation profiles/slope changed indicating upper airway narrowing? The HR and HRV status may also be reviewed. If so, then deliver therapy 2426 per programmed protocol:
Breath augmentation to restore normal MV and d_SaO2
Increase lung volume
Other therapies

In reviewing the fluctuations 2428, the algorithm may alternatively determine, for example, is there a reduction in lung volume? The HR and HRV status may also be reviewed. If so, then deliver therapy 2430 per programmed protocol:
Breath augmentation to restore normal MV and d_SaO2
Increase lung volume
Other therapies

After therapy completion, the algorithm may return to MONITOR MODE 2432.

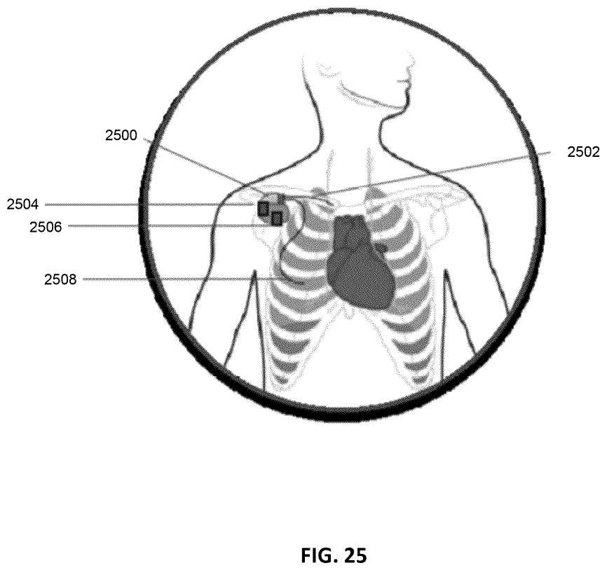

FIG. 25 shows one example of an implantable device 2500 which may include, for example, a phrenic nerve stimulation device 2502, thoracic pressure sensing 2508, impedance sensing, accelerometer 2504, and gyroscope 2506 inside the device. In one variation, the device may be a standalone device: The IPG and lead system can have multiple sensors to sense respiration and heart rate and correlate to these implantable sensors to polysomnography data. These sensors can include, e.g.,: 3-axis accelerometer 2504 (monitor chest movement during inspiration and exhalation), and gyroscope 2506 (monitor patient position during sleep and chest movement) inside the IPG to monitor chest and patient movement, impedance measurement between neurostim lead and IPG (monitor respiration be continuously measuring the lungs impedance during inhalation and exhalation and rest period), heart rate sensor (one or more electrodes integrated on neurostim lead to the device header or can). Heart rate variability (HRV) can be derived from heart rate sensor. The heart rate/ECG sensor can be configured with a single electrode on neurostimulation lead and the device can or another electrode in the IPG header. The heart rate sensor monitors the cardiac function and detect or predict normal and abnormal heart rhythms. The heart rate sensor can provide outputs such as normal heart rhythm and duration as well as abnormal heart rhythm, type (atrial fibrillation, tachycardia, bradycardia, ventricular arrythmia, or others) and duration of each. The timestamp and duration of arrythmias during sleep and awake can be reported.

The neurostimulation lead system could be used over a trial period to evaluate if a patient responds to prevention and mitigation of different types of hypoxia. The trial lead system could be implanted for multiple days & nights and then retrieved if the patient is not responding to therapies. The trial lead system could serve as chronic implant lead system.

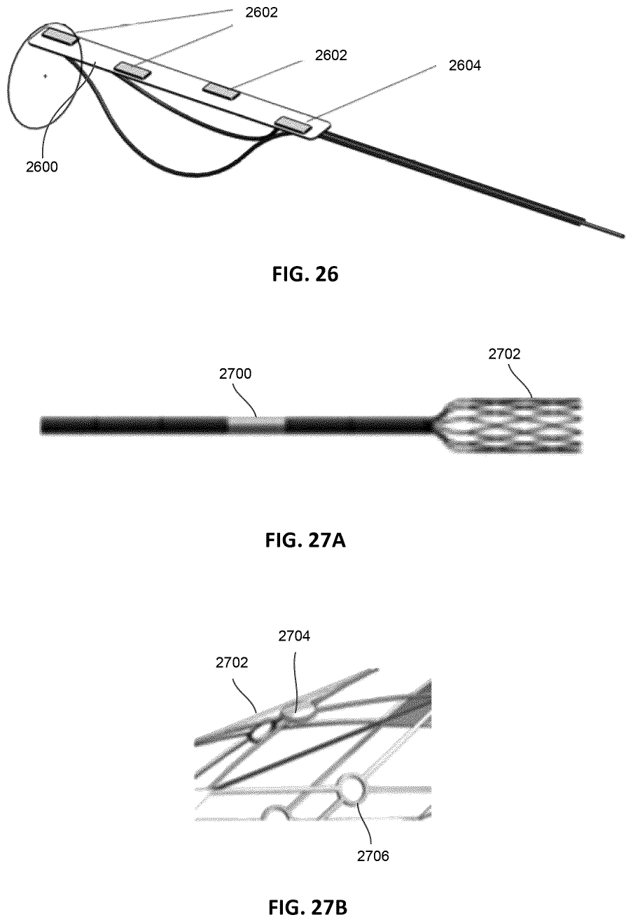

FIG. 26 shows another variation of a neurostimulation lead and heart rate sensor which includes one or more stimulation electrodes 2602 and one or more ECG/heart rate electrodes 2604 which may each be positioned upon a platform or substrate 2600. This variation may be used with the algorithms described herein.

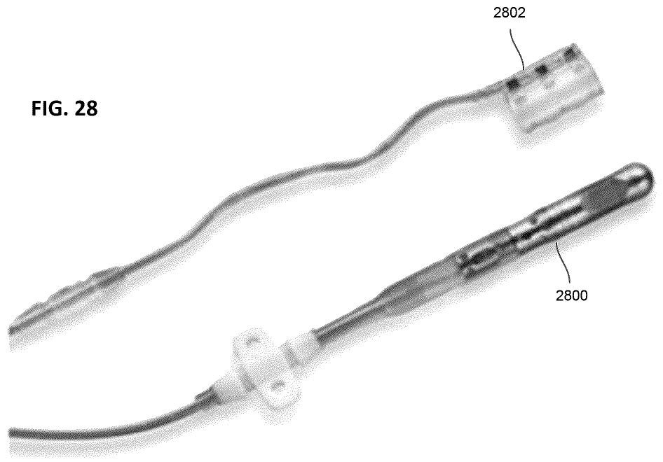

FIGS. 27A and 27B show an alternative neurostimulation lead and heart rate sensor and detail views of the sensors 2704, 2706 positioned along an expandable stent-like structure 2702 which may be attached to a catheter 2700. In addition to the above sensors 2704, 2706 or independently, the implantable system can have an implantable thoracic pressure sensor to monitor and/or measure respiration, as shown in FIG. 28, which illustrates a thoracic pressure sensor 2800 and cuff stimulation lead 2802.

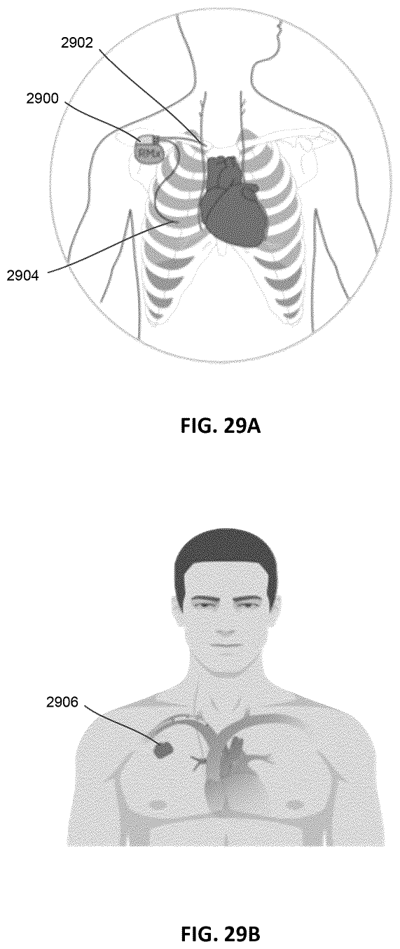

FIG. 29A illustrates an example of an implantable hypoglossal nerve stimulation 2900 with cuff stimulation lead 2902 and thoracic pressure sensor 2904 implanted within the patient body.

FIG. 29B illustrates another example of an implantable phrenic nerve stimulation 2906 with endovascular stimulation lead with sensing capabilities incorporated directly within the stimulation device itself.

In another aspect, phrenic nerve stimulation may be used to treat oxygen desaturation and T90. As discussed above, there are two main reasons for oxygen desaturation which sometimes leads to excessive hypoxia and high levels of T90. The d_SaO2 algorithm can be used to predict and detect reduction in oxygen saturation and classify as due to SBD or non-specific causes.

If the desaturation is a result of non-specific causes, the device can deliver therapies to increase SaO2 levels for a programmed period of time or as long as needed. Besides the d_SaO2 algorithm within the device, the device can communicate in real-time with, e.g., a finger pulse oximetry wearable patient can wear during the night. Real-time SaO2 monitoring with a wearable and adjusting the algorithm may be accomplished to achieve the baseline or normal therapeutic SaO2.

There could be several leading causes and sometimes a combination of two or three factors: (i) circulatory issues caused by reduction in blood-flow and cardiac output in heart failure, CAD, AFIB, or other cardiovascular patients; (ii) if desaturation (rapid desaturation is the signature according to publication/slide) is caused by obesity and visceral fat pushing against the diaphragm and lungs and causing reduction in end-reserve-volume (ERV) or lung volume or hypoventilation syndrome (easily measurable by monitoring respiratory rate); (iii) changes in respiration and air flow. The MV may be modulated or normalized by maintaining SaO2 with a stable range for a specific patient. Reduction in MV could lead to changes in SaO2 and eventually hypoxia. MV modulation can be accomplished by PNS with variety of stimulation modalities including breath augmentation, increasing LV, entrainment, combination of airway management and entrainment, or airway management and augmentation.

Neurostimulation systems typically include one or more electrode carrying neurostimulation leads, which are positioned temporarily or implanted at a desired stimulation site. A neurostimulation device such as an implantable pulse generator can be implanted remotely from the stimulation site and coupled to the neurostimulation leads in order to deliver electrical pulses through the neurostimulation leads to stimulate the tissue.

Hypoxia is also believed to contribute to the development of heart failure, particularly through hypertension.

Oxygen desaturations at night, changes in intrathoracic pressure, and arousals may adversely affect cardiac function and eventually result in an imbalance between myocardial oxygen delivery and consumption. In heart failure patients with hypoxia, there is believed to be an increased incidence of atrial fibrillation, ventricular arrhythmias and low left ventricular ejection fraction. Atrial fibrillation may be caused in part by increased right heart afterload due to hypoxic vasoconstriction which produces pulmonary hypertension. Periodic breathing such as Cheyne-Stokes associated with CSA, create wide fluctuations in intrathoracic pressure and SaO2 leading hypoxia. The untreated hypoxia may trigger a negative chain of events that leads to worsening of heart failure.

Accordingly it would be desirable to treat and minimize hypoxia as result of sleep apnea or cardiovascular issues in heart failure patients to reduce the negative effects of hypoxia on the patient's disease status.

In yet another aspect, nocturnal hypoxia burden is the cumulative exposure to hypoxia experienced overnight during sleep which may contribute to pathophysiology of the cardiovascular (CV) disease and CV death by increasing the production of reactive oxygen species, vascular inflammation, autonomic imbalance, activation of the sympathetic nervous system, and elevating blood pressure.

In overnight sleep studies or polysomnography, oxygen saturation is one of the several parameters measured, e.g., by wearing a finger pulse oximetry device. The number of episodes in which oxygen saturation falls below 90% is reported as ODI (Oxygen Desaturation Index). One of the shortcomings of ODI is the omission of measuring and reporting the amount of time a patient spent below an oxygen saturation level of 90%. Polysomnography also reports AHI (Apnea Hypopnea Index) which is the number of episodes per hours of sleep. One of the main goals of current sleep apnea therapies (e.g., CPAP, dental appliances, hypoglossal nerve stimulators) is to reduce AHI and ODI while improving quality of life.

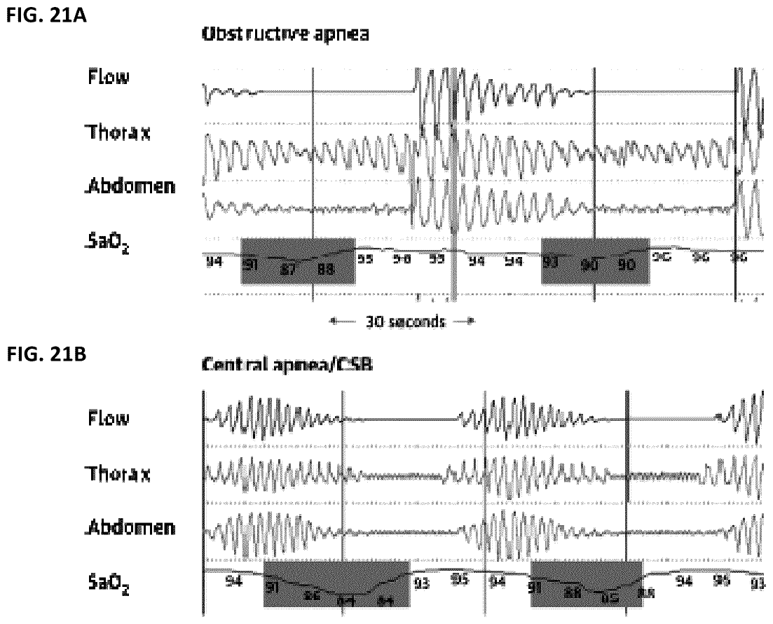

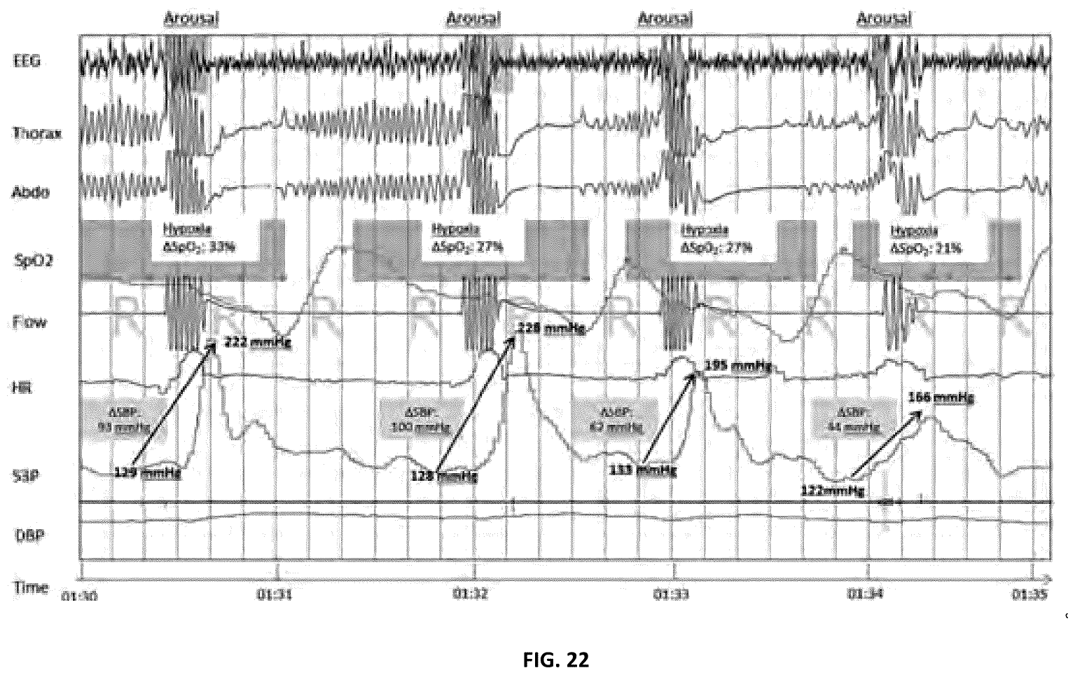

FIG. 21A shows an example of the various signals measured during a sleep study for a patient with OSA and FIG. 21B shows an example of the measured signals for a patient with CSA. Both charts show changes in oxygen saturation (SaO2) during apneic episodes showing airflow (“Flow”), thoracic and abdominal wall movements (“Thorax” and “Abdomen”), and PaO2 (partial pressure of arterial oxygen). Desaturation is delayed in FIG. 21B for the patient with CSA because of relatively longer circulation times in heart failure.

In recent clinical studies in cardiovascular patients with sleep apnea, the researchers have identified hypoxia as a new cardiovascular risk biomarker for mortality and comorbidities including incidence of AFIB and hypertension. In all these clinical trials, the AHI did not present as a statistically significant biomarker for cardiovascular risk. In multiple clinical studies in patients with sleep disorder breathing (SDB), atrial fibrillation (AFIB), coronary artery bypass surgery (CABG), heart failure, pulmonary hypertension, and treatment emergent CSA, hypoxia was a statistically significant biomarker for cardiovascular mortality and comorbidities.

Hypoxia is a state in which oxygen is not available in sufficient amounts at the tissue level to maintain adequate homeostasis; this can result from inadequate oxygen delivery to the tissues either due to low blood supply or low oxygen content in the blood (hypoxemia). As described, research points to nocturnal hypoxia and hypoxemia as independent risk factor for cardiovascular mortality and morbidity among patients with sleep disorder breathing, cardiovascular disease such as AFIB, heart failure, obesity, and COPD (chronic obstructive pulmonary disorder).

To define and diagnose patients at risk of hypoxia, medical societies have created a new risk factor, T90, which relates to the time duration a patient spends under 90% oxygen saturation level during sleep. It is primarily calculated by the total time duration when oxygen saturation is less than 90% divided by total sleep time.

In contrast, sleep studies report that when episodes or incidence of ODI (oxygen desaturation per hours of sleep) is reported, the duration which a patient is desaturated is not reported. These publications do not identify AHI or ODI as independent cardiovascular and mortality risk factor. However, the T90 level is identified as a mortality and cardiovascular independent risk factor.

T90 has multiple root causes and is divided into two categories: T90_{desat} and T90ₙₛ (non-specific). A study of 2840 men with 8.8 years follow-up, 80% of T90 due to desaturation while another 20% were non-specific. This study also identified T90 as an independent mortality risk factor (European heart journal 2020 41:533-541). As discussed, T90_{desat} is caused by acute oxygen desaturation due to SDB including central, obstructive, and other types of disordered breathing during sleep. Furthermore, T90ₙₛ could have multiple causes including poor circulation primarily in cardiovascular and heart failure patients, obesity which leads to obesity hypoventilation syndrome, reduction in lung volume due to visceral fat, and COPD. The combination of COPD and obesity further exacerbates T90ₙₛ levels.

FIG. 22 shows a chart illustrating significant reduction of the airflow (“Flow”) over time leading to a progressively declining level of oxygen saturation (“SpO2”). As a consequence, dramatic increases of systolic blood pressure (“SBP”) up to 100 mmHg are possible. The corresponding diastolic blood pressure (“DBP”); electroencephalogram (“EEG”); heart rate (“HR”); nocturnal blood pressure fluctuations (“NBPF”); systolic blood pressure (“SBP”) are also shown. The sudden and repetitive changes in HR and SBP due to hypoxia pose potential long-term cardiovascular impact.

For comparison, existing treatments and outcomes are described. Continuous Positive Airway Pressure (CPAP) is considered the primary choice for treatment of sleep apnea and the associated impacts. Recently, there has been additional focus and clinical studies regarding the impact of sleep apnea and CPAP therapy on cardiovascular outcomes. The CERCAS (Effect of Continuous Positive Airway Pressure on Hypertension and Cardiovascular Morbidity-Mortality in Patients with Sleep Apnea and no Daytime Sleepiness) trial randomized 725 non-sleepy patients with moderate or severe OSA to CPAP or no active intervention, after excluding those with previous CVD events. CPAP did not result in a reduction in the incidence of hypertension or CVD events during 3 years' follow-up, although a post hoc secondary analysis showed that adherence to CPAP for more than 4 h per night did reduce both endpoints. Patient compliance has been the primary issue with CPAP.

In other RCTs (Randomized Clinical Trials) for treatment of apnea and cardiovascular impact, no statistically significant impact on CV endpoint improvement has been observed.

The SAVE (Continuous Positive Airway Pressure Treatment of Obstructive Sleep Apnea to Prevent Cardiovascular Disease) trial randomized over 2,000 patients with established CV or cerebrovascular disease to CPAP plus usual care, or usual care alone. An effort was made to familiarize patients with CPAP using a run-in period. The use of CPAP (over a mean follow-up of 43 months) did not significantly reduce the primary composite endpoint of MACE, although sleepiness did improve. In a prespecified subgroup analysis, those with CPAP adherence >4 h per night had a lower risk of stroke (HR: 0.56; 95% CI: 0.32-1.00) and total cerebrovascular events (HR: 0.52; 95% CI: 0.30-0.90).

In the RICCADSA (Continuous Positive Airway Pressure [CPAP] Treatment in Coronary Artery Disease and Sleep Apnea) trial, patients with moderate or severe OSA (but with no daytime sleepiness) were randomized to CPAP or control after coronary revascularization and followed up for a mean of 57 months. The CPAP group showed no change in CVD endpoints including repeat revascularization, although the subgroup using CPAP >4 h per night had a lower CV risk (HR: 0.29; 95% CI: 0.10-0.86).

A further study, the ISAACC (Continuous Positive Airway Pressure (CPAP) in Patients With Acute Coronary Syndrome and Obstructive Sleep Apnea (OSA)) trial examined patients with acute coronary syndrome and moderate or severe OSA. CPAP had no significant effect on the primary composite end point of CVD events or death. In this trial, there was no relationship between hours of CPAP use and out-comes, although median adherence to CPAP was low at 2.78 h per night. CPAP users did have an improvement in hypertension control and daytime sleepiness, but not in quality of life.

Adaptive Servo Ventilation (ASV) is considered to increase inspiratory support during hypopnea, withdraws support during hyperventilation, provides mandatory breaths during apnea, and generates background positive airway pressure. It is therefore effective in both CSA and OSA.

However, the SERVE-HF (Treatment of Predominant Central Sleep Apnea by Adaptive Servo Ventilation in Patients With Heart Failure) trial, a large RCT to assess the impact of ASV on hospitalization, life-saving cardiovascular intervention, or death in those with HF and (central sleep apnea (CSA) in patients with a LV ejection fraction >=45% and moderate-to-severe CSA reported no difference between the 2 groups, despite a powerful effect on AHI. Surprisingly, there was a higher all-cause and cardiovascular mortality in those treated with ASV, largely driven by an increase in sudden death. Various explanations have been proposed including a direct toxic effect of ASV on patients with poor LV function and a low pulmonary capillary wedge pressure; or that CSA may be at least partially adaptive for patients with severe HF.

In the meantime, the use of ASV (or perhaps other airway pressure therapies) for the treatment of predominantly CSA in HFrEF cannot be recommended.

In addition to positive pressure therapies, weight loss, bariatric surgery, oxygen therapy, and pharmaceuticals have not shown a significant impact on the CV endpoints and outcomes.

Phrenic nerve stimulation to treat CSA has shown to treat majority of CSA episodes and improve oxygen saturation. However, the device did not treat the OSA episodes in the same patients and was not powered to measure CV endpoints.

Thus, there is no current viable therapy to directly treat both forms of hypoxia and the cardiovascular outcomes. We believe our inventions provide an opportunity to treat the root cause of cardiovascular risks in patients experiencing nocturnal hypoxia whether from desaturation caused by apnea or non-specific causes.

The applications of the devices and methods discussed above are not limited to treatments for disordered breathing but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

As a person skilled in the art will recognize from the previous detailed description and figures that modifications and changes may be made to the preferred embodiments of the invention without departing from the scope of this invention defined in the following claims.

What is claimed is:

1. A system for treating nocturnal hypoxia, comprising:
one or more sensors configured to be implanted within a subject,
a controller in communication with the one or more oximetry sensors, wherein the controller is programmed to predict a change in oxygen saturation indicative of hypoxia in the subject by identifying one or more subject-specific features from a polysomnography of the subject and correlating the one or more subject-specific features to data received from the one or more sensors; and
one or more electrodes in communication with the controller, wherein the one or more electrodes are configured for placement against tissue associated with respiration of the subject and are further configured to deliver a stimulation to the tissue in response to the predicted change in oxygen saturation.

2. The system of claim 1 wherein the one or more sensors comprise an accelerometer, gyroscope, thoracic impedance, thoracic pressure, or heart rate sensors.

3. The system of claim 1 further comprising one or more polysomnography sensors.

4. The system of claim 3 wherein the one or more polysomnography sensors comprise pulse oximetry, EEG, respiratory flow, ECG, or jaw movement sensors.

5. The system of claim 1 wherein the controller is further programmed to monitor for a fluctuation in one or more parameters including a breathing rate, tidal volume, respiratory flow, oxygen saturation, electrocardiogram, heart rate, or heart rate variability.

6. The system of claim 1 wherein the controller is further programmed to detect for hypoventilation or a reduced tidal volume in the subject having COPD.

7. The system of claim 6 wherein the controller is further programmed to provide the stimulation to the subject for initiating entrainment, breath augmentation, or lung volume increase in the subject.

8. The system of claim 1 wherein the controller is further programmed to detect for hypoventilation in the subject having obesity.

9. The system of claim 8 wherein the controller is further programmed to provide the stimulation to the subject for initiating entrainment in the subject.

10. The system of claim 1 wherein the controller is further programmed to detect for hypoventilation in the subject having CHF.

11. The system of claim 10 wherein the controller is further programmed to provide the stimulation to the subject for initiating entrainment or increasing a lung volume in the subject.

12. The system of claim 1 wherein the controller is further programmed to detect for an inspiration and/or exhalation profile change in the subject.

13. The system of claim 11 wherein the controller is further programmed to provide the stimulation to the subject for initiating a breath augmentation in the subject.

14. The system of claim 1 wherein the controller is further programmed to detect for a reduction in lung volume in the subject.

15. The system of claim 14 wherein the controller is further programmed to provide the stimulation to the subject for initiating a breath augmentation in the subject.

16. A method for treating nocturnal hypoxia, comprising:
monitoring respiration and heart rate within a phrenic nerve stimulation device implanted within a subject;
predicting a potential reduction in oxygen saturation during sleep of the subject via a controller programmed to predict a change in oxygen saturation indicative of hypoxia in the subject by identifying one or more patient-specific features; and
stimulating a phrenic nerve and/or diaphragm via one or more electrodes in communication with the controller for a period of time in response to the predicted change until an oxygen saturation level within the subject is normalized and hypoxia is mitigated.

17. The method of claim 16 wherein monitoring respiration and heart rate comprises monitoring via one or more sensors comprising an accelerometer, gyroscope, thoracic impedance, thoracic pressure, or heart rate sensors.

18. The method of claim 16 wherein monitoring respiration and heart rate comprises monitoring via one or more sensors comprising polysomnography sensors.

19. The method of claim 18 wherein the one or more polysomnography sensors comprise pulse oximetry, EEG, respiratory flow, ECG, or jaw movement sensors.

20. The method of claim 16 wherein predicting the potential reduction in oxygen saturation comprises predicting the potential reduction based on a fluctuation in one or more parameters including a breathing rate, tidal volume, respiratory flow, oxygen saturation, electrocardiogram, heart rate, or heart rate variability.

21. The method of claim 16 wherein predicting the potential reduction in oxygen saturation comprises predicting the potential reduction based on a presence of hypoventilation or a reduced tidal volume in the subject having COPD.

22. The method of claim 21 wherein stimulating the phrenic nerve comprises providing a stimulation to the subject for initiating entrainment, breath augmentation, or lung volume increase in the subject.

23. The method of claim 16 wherein predicting the potential reduction in oxygen saturation comprises predicting the potential reduction based on a presence of hypoventilation in the subject having obesity.

24. The method of claim 23 wherein stimulating the phrenic nerve comprises providing a stimulation to the subject for initiating entrainment in the subject.

25. The method of claim 16 wherein predicting the potential reduction in oxygen saturation comprises predicting the potential reduction based on a presence of hypoventilation in the subject having CHF.

26. The method of claim 25 wherein stimulating the phrenic nerve comprises providing a stimulation to the subject for initiating entrainment or increasing a lung volume in the subject.

27. The method of claim 16 wherein predicting the potential reduction in oxygen saturation comprises predicting the potential reduction based on a presence of an inspiration and/or exhalation profile change in the subject.

28. The method of claim 27 wherein stimulating the phrenic nerve comprises providing a stimulation to the subject for initiating a breath augmentation in the subject.

29. The method of claim 16 wherein predicting the potential reduction in oxygen saturation comprises predicting the potential reduction based on a presence of a reduction in lung volume in the subject.

30. The method of claim 29 wherein stimulating the phrenic nerve comprises providing a stimulation to the subject for initiating a breath augmentation in the subject.
